(19) United States
(12) Patent Application Publication
(10) Pub. No.: US 2025/0281648 A1
(43) Pub. Date: Sep. 11, 2025

(54) USE OF TRANSDIFFERENTIATION OF GLIAL CELLS INTO NEURONS IN PREVENTION OR TREATMENT OF DISEASES ASSOCIATED WITH NEURON LOSS-OF-FUNCTION OR DEATH

(71) Applicant: SHANGHAI GENEMAGIC BIOSCIENCES CO., LTD., Shanghai (CN)

(72) Inventors: Haibo ZHOU, Shanghai (CN); Xinde HU, Shanghai (CN); Jinlin SU, Shanghai (CN)

(21) Appl. No.: 18/264,923

(22) PCT Filed: Feb. 10, 2022
(86) PCT No.: PCT/CN2022/075823

§ 371 Date: Aug. 9, 2023

(30) Foreign Application Priority Data
Feb. 10, 2021 (CN) 202110185323.2

(51) Int. Cl. A61K 48/00; C12N 9/22; C12N 15/11

(52) U.S. Cl. CPC A61K 48/0075; A61K 48/005; C12N 9/226; C12N 15/11; C12N 2310/20

(57) ABSTRACT

The transdifferentiation of glial cells into neurons is useful for prevention or treatment of a disease associated with loss of function or death. The present disclosure relates to the field of biomedicines. More specifically, the present disclosure relates to use of a REST inhibitor in the treatment of a disease associated with loss of function or death of neurons. The present disclosure can effectively induce transdifferentiation of astrocytes into dopamine neurons by inhibiting the expression, content or activity of a gene of REST or an RNA thereof or an encoding protein thereof in astrocytes in the brain, and can effectively induce transdifferentiation of Müller glia (MG) into retinal ganglion cells (RGCs) or photoreceptor cells by inhibiting the expression, content or activity of the gene of REST or the RNA thereof or the encoding protein thereof in the retina, thereby preventing and/or treating the disease associated with loss of function or death of neurons.

### RELATED APPLICATIONS

The present application claims the benefit of the application No. 202110185323.2 entitled “USE OF TRANSDIFFERENTIATION OF GLIAL CELLS INTO NEURONS FOR PREVENTION OR TREATMENT OF DISEASE ASSOCIATED WITH LOSS OF FUNCTION OR DEATH OF NEURONS” filed on Feb. 10, 2021, which is incorporated herein by reference in its entirety.

### TECHNICAL FIELD

The present disclosure relates to the field of biomedicines. More specifically, the present disclosure relates to use of an REST (REI-silencing transcription factor) inhibitor in the prevention and/or treatment of a disease associated with loss of function or death of neurons.

### BACKGROUND

Parkinson's disease (PD) is a disease associated with loss of function or death of neurons, characterized by loss of dopamine neurons in the substantia nigra of the midbrain. Currently, the main treatment for Parkinson's disease is a drug represented by levodopa. Meanwhile, the symptoms can be improved to a certain extent by surgical treatment. It should be noted that all these approaches can only partially alleviate the disease conditions, but they cannot prevent the progression of the disease.

Müller glia (MG) are the major neuroglial cells in retinal tissue. In zebrafish, when the retina is damaged, Müller glia can proliferate and differentiate into photoreceptor cells, retinal ganglion cells (RGCs), bipolar cells and other retinal cells, which help to repair the damage. However, in higher mammals, MG loses its ability to differentiate into various functional cells of the retina after retinal maturation. Photoreceptor cells are a special type of nerve cells in the retina and are the only cells in the visual system that capture light signals. Retinitis pigmentosa, congenital amaurosis (LCA), age-related macular degeneration, diabetic retinopathy and other ophthalmic diseases can cause the death of photoreceptor cells, thereby causing blindness. The retinal ganglion cells are nerve cells located in the innermost layer of the retina, and their dendrites are mainly connected with bipolar cells, and their axons extend to the optic papilla to form optic nerve. Degeneration of retinal ganglion cells (RGCs) is the major cause of permanent blindness. RGCs are the only efferent neurons of the retina, and thus degeneration of RGCs may lead to retinal diseases leading to permanent blindness. Therefore, reconstituting functional photoreceptor cells or RGCs helps to restore vision and is a potential therapeutic approach to restoring visual function.

There remains an urgent need in the art to develop new targets and new therapies that can effectively treat a disease associated with loss of function or death of neurons.

### SUMMARY

One objective of the present disclosure is to provide a method for producing functional dopamine neurons from glial cells.

Another objective of the present disclosure is to provide use of a REST inhibitor in the prevention and/or treatment of a disease associated with loss of function or death of functional dopamine neurons.

Yet another objective of the present disclosure is to provide a method for producing functional retinal ganglion cells (RGCs) or photoreceptor cells from Müller glia (MG).

Still another objective of the present disclosure is to provide use of a REST inhibitor in the prevention and/or treatment of a visual system disease associated with loss of function or death of RGCs or photoreceptor cells.

In one embodiment, the present disclosure provides a method for producing functional dopamine neurons from glial cells, which comprises transdifferentiating or reprogramming the glial cells into functional dopamine neurons by using a REST inhibitor, wherein the REST inhibitor reduces the expression, content, or activity of a gene of REST, an RNA thereof, or an encoding protein thereof.

In a preferred embodiment, the glial cells are selected from astrocytes, oligodendrocytes, ependymocytes, Schwann cells, NG2 cells, satellite cells, or a combination thereof.

In a more preferred embodiment, the glial cells are astrocytes.

In a preferred embodiment, the astrocytes are from central nervous system, comprising striatum, substantia nigra, ventral tegmental area of a midbrain, spinal cord, hypothalamus, dorsal midbrain, or cerebral cortex.

In a more preferred embodiment, the astrocytes are from the striatum and the substantia nigra.

In another embodiment, the present disclosure provides the use of a REST inhibitor in the preparation of a medicament for preventing and/or treating a disease associated with loss of function or death of functional dopamine neurons, wherein the REST inhibitor reduces the expression, content or activity of a gene of REST, an RNA thereof, or an encoding protein thereof.

In a preferred embodiment, the medicament is formulated for administration to a central nervous system, comprising striatum, substantia nigra, ventral tegmental area of midbrain, spinal cord, hypothalamus, dorsal midbrain or cerebral cortex, etc.

In a more preferred embodiment, the medicament is formulated for administration to the striatum and the substantia nigra.

In a preferred embodiment, the disease associated with loss of function or death of the functional dopamine neurons is a nervous system disease, which comprise stroke, Parkinson's disease, schizophrenia, and depression.

In a more preferred embodiment, the disease associated with loss of function or death of functional dopamine neurons is Parkinson's disease.

In another embodiment, the present disclosure provides a method for producing functional retinal ganglion cells (RGCs) or photoreceptor cells from Müller glia, which comprises transdifferentiating or reprogramming Müller glia into functional RGCs or photoreceptor cells by using a REST inhibitor, wherein the REST inhibitor reduces the expression, content, or activity of a gene of REST, an RNA thereof, or an encoding protein thereof.

In a preferred embodiment, the Müller glia are from retina.

In a preferred embodiment, the photoreceptor cells comprise rod cells and cone cells.

In another embodiment, the present disclosure provides use of a REST inhibitor in the preparation of a medicament for preventing and/or treating a visual system disease associated with loss of function or death of RGCs or photoreceptor cells, wherein the REST inhibitor reduces the expression, content, or activity of a gene of REST, an RNA thereof, or an encoding protein thereof.

In a preferred embodiment, the medicament is formulated for administration to visual system.

In a more preferred embodiment, the medicament is formulated for administrating to subretinal space or vitreous cavity, wherein the medicament plays its role by acting on Müller glia.

In a preferred embodiment, the nervous system disease associated with loss of function or death of RGCs is selected from: visual impairment due to death of RGCs, glaucoma, age-related RGC pathology, optic nerve damage, retinal ischemia or hemorrhage, Leber hereditary optic neuropathy, or a combination thereof.

In a preferred embodiment, the visual system disease associated with loss of function or death of photoreceptor cells is selected from: photoreceptor degeneration or death due to damage or degenerative diseases, macular degeneration, retinitis pigmentosa, diabetes-related blindness, night blindness, color blindness, inherited blindness, congenital amaurosis, or the combination thereof.

In a preferred embodiment, the REST inhibitor is selected from: antibodies, small molecule compounds, microRNA, siRNA, shRNA, antisense oligonucleotides, REST binding proteins and protein domains, polypeptides, aptamers, gene editors, PROTACs, epigenetic regulators, or a combination thereof.

In a preferred embodiment, the REST inhibitor comprises:
(a) a gene-editing protein or an expression vector thereof, an editing system comprising: a CRISPR system (including a CRISPR/dCas system), a ZFN system, a TALEN system, an RNA-editing system, or a combination thereof, and (b) one or more gRNAs or an expression vector thereof, wherein the gRNA is a DNA or an RNA guiding the gene-editing protein to specifically bind to a REST gene, wherein, an RNA-targeting gene-editing protein and an RNA-targeting gRNA are preferable.

In a preferred embodiment, the gRNA comprises a sequence complementary to a target sequence.

In a preferred embodiment, the gRNA guides the gene-editing protein to specifically bind to nucleotides at positions 867-1103 (SEQ ID NO: 3) of REST coding sequence.

In a preferred embodiment, the gRNA comprises a sequence complementary to SEQ ID NO: 3.

In a preferred embodiment, the gRNA comprises a sequence selected from SEQ ID NOs: 4-20 and 83-118 or comprises a sequence encoded by sequences set forth in SEQ ID NOs: 55-62 and 71-76, and preferably, the gRNA comprises a sequence selected from SEQ ID NOs: 10 and 93-103.

In a preferred embodiment, the gRNA comprises a sequence fully complementary to a target sequence, or comprises a complementary sequence having no more than 3 base mismatches to the target sequence.

In a preferred embodiment, the gRNA and target sequence belong to a same species or different species.

In a preferred embodiment, the gRNA and target sequence are from human, cynomolgus monkey, or mouse.

In another embodiment, the present disclosure provides a pharmaceutical composition or a package of drug or a kit comprising a REST inhibitor.

In a preferred embodiment, the REST inhibitor is selected from: antibodies, small molecule compounds, microRNA, siRNA, shRNA, antisense oligonucleotides, REST binding proteins or protein domains, polypeptides, aptamers, gene editors, PROTACs, epigenetic regulators, or a combination thereof.

In a preferred embodiment, the REST inhibitor comprises:
a gene-editing protein or an expression vector thereof, and
one or more gRNAs or an expression vector thereof, wherein the gRNA is a DNA or an RNA guiding the gene-editing protein to specifically bind to a REST gene.

In a preferred embodiment, an editing system comprises: a CRISPR system (including a CRISPR/dCas system), a ZFN system, a TALEN system, an RNA-editing system, or a combination thereof.

In a preferred embodiment, the gene-editing protein is an RNA-targeting gene-editing protein.

In a preferred embodiment, the gRNA is an RNA-targeting gRNA.

In a preferred embodiment, the gRNA comprises a sequence complementary to a target sequence.

In a preferred embodiment, the gRNA guides the gene-editing protein to specifically bind to nucleotides at positions 867-1103 (SEQ ID NO: 3) corresponding to a REST coding sequence.

In a preferred embodiment, the gRNA comprises a sequence complementary to SEQ ID NO: 3.

In a preferred embodiment, the gRNA comprises a sequence selected from SEQ ID NOs: 4-20 and 83-118 or comprises a sequence encoded by sequences set forth in SEQ ID NOs: 55-62 and 71-76, and preferably, the gRNA comprises a sequence selected from SEQ ID NOs: 10 and 93-103.

In a preferred embodiment, the gRNA comprises a sequence fully complementary to a target sequence, or comprises a complementary sequence having no more than 3 base mismatches to the target sequence.

In a preferred embodiment, the gRNA and target sequence belong to a same species or different species.

In a preferred embodiment, the gRNA and target sequence are from human, cynomolgus monkey, or mouse.

In a preferred embodiment, the pharmaceutical composition or the package of drug or the kit further comprises a vector or carrier for delivery of the REST inhibitor.

In a preferred embodiment, the vector or carrier is a viral vector, a liposome, a nanoparticle, an exosome, or a virus-like particle, preferably AAV.

In a preferred embodiment, the RNA-targeting gene-editing protein is selected from: Cas13d, CasRx, Cas13X, Cas13a, Cas13b, Cas13c, Cas13Y, and functional domains thereof.

In a more preferred embodiment, the RNA-targeting gene-editing protein is selected from: CasRx, Cas13X, and Cas13Y.

In a more preferred embodiment, the RNA-targeting gene-editing protein is CasRx.

In a preferred embodiment, the pharmaceutical composition or the package of drug or the kit comprises only a single type of gRNA or 2, 3, 4, 5, 6 different gRNAs targeting a REST mRNA sequence.

In another preferred embodiment, the gRNA expression vector encodes only a single type of gRNA or 2, 3, 4, 5, 6 different gRNAs targeting a REST mRNA sequence.

In a preferred embodiment, the expression vector comprises:
a nucleotide sequence encoding the gene-editing protein, the nucleotide sequence being operably linked to a promoter leading to expression of the gene-editing protein, and
at least one nucleotide sequence encoding a gRNA targeting a REST mRNA sequence, the nucleotide sequence being operably linked to a promoter leading to expression of the gRNA in mammalian cells, e.g., a U6 promoter.

In a more preferred embodiment, the promoter is a glial cell-specific promoter or a MG-specific promoter.

In a more preferred embodiment, the glial cell-specific promoter is selected from a GFAP promoter, an ALDH1L1 promoter, an EAAT1/GLAST promoter, a glutamine synthetase promoter, an S100β promoter, and an EAAT2/GLT-1 promoter, or the MG cell-specific promoter is selected from a GFAP promoter, an ALDH1L1 promoter, a Glast (also known as Slcla3) promoter, and an Rlbp1 promoter.

In a preferred embodiment, the expression vector is comprised in a nanoparticle.

In a preferred embodiment, the expression vector is a gene therapy vector.

In a more preferred embodiment, the gene therapy vector is a viral gene therapy vector.

In a more preferred embodiment, the expression vector is a viral vector selected from: an adeno-associated viral (AAV) vector, a recombinant adeno-associated viral (rAAV) vector, an adenoviral vector, a lentiviral vector, a retroviral vector, herpesvirus, an SV40 vector, a poxvirus vector, and a combination thereof.

In a more preferred embodiment, the expression vector is an AAV vector or an rAAV vector.

In a preferred embodiment, the composition is locally administered to at least one of the followings: 1) glial cells in retina: ii) glial cells in striatum, preferably glial cells in putamen: iii) glial cells in substantia nigra: iv) glial cells in inner ear: v) glial cells in spinal cord: vi) glial cells in prefrontal cortex: vii) glial cells in motor cortex: viii) glial cells in ventral tegmental area (VTA); and ix) glial cells in hypothalamus.

In a preferred embodiment, the pharmaceutical composition or the package of drug or the kit further comprises i) one or more dopamine neuron-associated factors, or ii) at least one expression vector for expressing the one or more dopamine neuron-associated factors in the glial cells.

In a preferred embodiment, the one or more dopamine neuron-associated factors are selected from: Lmx1a, Lmx1b, FoxA2, Nurr1, Pitx3, Gata2, Gata3, FGF8, BMP, En1, En2, PET1, Pax family proteins, SHH, Wnt family proteins, and TGF-β family proteins.

In a preferred embodiment, the pharmaceutical composition or the package of drug or the kit further comprises: i) one or more factors selected from β-catenin, Oct4, Sox2, Klf4, Crx, Brn3a, Brn3b, Math5, Nr2e3, and Nr1, and/or ii) at least one expression vector for expressing one or more factors selected from β-catenin, Oct4, Sox2, Klf4, Crx, Brn3a, Brn3b, Math5, Nr2e3, and Nr1 in glial cells.

In a preferred embodiment, the composition is further formulated for injection, intracranial administration, intraocular administration, inhalation, parenteral administration, intravenous administration, intramuscular administration, intradermal administration, epidermal administration, or oral administration.

In a preferred embodiment, the AAV vector comprises:
a nucleotide sequence encoding the gene-editing protein, the nucleotide sequence being operably linked to a promoter leading to expression of the gene-editing protein in glial cells; and
at least one nucleotide sequence encoding a gRNA targeting a REST mRNA sequence, the nucleotide sequence being operably linked to a promoter leading to expression of the gRNA in mammalian cells, e.g., a U6 promoter.

In a more preferred embodiment, the promoter is a glial cell-specific promoter or a MG-specific promoter.

In a more preferred embodiment, the glial cell-specific promoter is selected from a GFAP promoter, an ALDH1L1 promoter, an EAAT1/GLAST promoter, a glutamine synthetase promoter, an S100β promoter, and an EAAT2/GLT-1 promoter.

In a more preferred embodiment, the MG cell-specific promoter is selected from a GFAP promoter, an ALDH1L1 promoter, a Glast (also known as Slcla3) promoter, and an Rlbp1 promoter.

In a preferred embodiment, the efficiency of transdifferentiation of the glial cells is at least 1%, or at least 10%, 20%, 30%, 40%, or 50%.

In another preferred embodiment, the disease associated with loss of function or death of neurons is selected from: Parkinson's disease, schizophrenia, depression, vision impairment due to death of RGCs, glaucoma, age-related RGC pathology, optic nerve damage, retinal ischemia or hemorrhage, Leber hereditary optic neuropathy, photoreceptor cell degeneration or death due to damage or degenerative diseases, macular degeneration, retinitis pigmentosa, diabetes-related blindness, night blindness, color blindness, inherited blindness, congenital amaurosis, or a combination thereof.

In another preferred embodiment, the RGCs can be integrated into the visual pathway and improve visual function.

In another preferred embodiment, the RGCs can achieve functional projection to the central visual region and improve visual function.

In another preferred embodiment, improving visual function is to improve visual function in a mammal suffering from a retinal disease caused by neurodegeneration.

In another preferred embodiment, the MG cells are transdifferentiated into RGC cells and also into axon-free cells.

In another preferred embodiment, an RGC (1) expresses Brn3a, Rbpms, Foxp2, Brn3c, and/or parvalbumin: (2) is F-RGC, RGC type 3, or PV-RGC: (3) is integrated into an existing retinal pathway in the subject (e.g., central information can be projected to dLGN, and vision can be partially restored by relaying visual information to V1); and/or (4) can receive visual information characterized in that during light stimulation or synaptic connections (for example, with existing functional dLGN neurons in the brain), presynaptic neurotransmitters can produce a biological reaction or generate action potentials.

In another preferred embodiment, dopamine neurons (1) express tyrosine hydroxylase (TH), dopamine transporter (DAT), vesicular monoamine transporter 2 (VMAT2), engrailed homeobox 1 (En1), FoxA2, and/or LIM homeobox transcription factor 1 alpha (Lmx1a): (2) perform the synthesis and release of presynaptic neurotransmitters: (3) are integrated into an existing neuronal circuit in the brain of the subject; and/or (4) are characterized by its ability to establish action potentials, synaptic connections, biogenesis of presynaptic neurotransmitters and/or postsynaptic responses.

In another preferred embodiment, the plurality of neuroglial cells in the striatum are reprogrammed or transdifferentiated, and at least 1% of the glial cells are converted into dopamine neurons.

In another preferred embodiment, the mammal comprises a mammal suffering from a disease associated with loss of function or death of neurons.

In another preferred embodiment, the mammal comprises human or non-human mammal.

In another preferred embodiment, the non-human mammal comprises rodent (e.g., mouse, rat, or rabbit), and primate (e.g., monkey).

In another preferred embodiment, the gene editors are driven by a neuroglial cell-specific promoter (e.g., a GFAP promoter) for expression.

In another preferred embodiment, the gene editor comprises 1 or more gRNAs and gene-editing proteins.

In another preferred embodiment, the gRNA guides the gene-editing protein to specifically bind to the RNA of the REST gene.

In another preferred embodiment, the gRNA guides the gene-editing protein to specifically bind to the mRNA of the REST gene.

In another preferred embodiment, the nucleotide sequence of the gRNA is, for example, set forth in SEQ ID NOs: 4-20, preferably SEQ ID NO: 10.

In another preferred embodiment, the source of the gene-editing protein is selected from: Streptococcus pyogenes, Staphylococcus aureus, acidaminococcus sp, Lachnospiraceae bacterium, Ruminococcus flavefaciens, or a combination thereof.

In another preferred embodiment, the REST is from a mammal; preferably, it is from human, monkey, mouse, rat, or rabbit: more preferably, it is from human.

In another preferred embodiment, the REST gene comprises a wild-type REST gene and a mutant-type REST gene.

In another preferred embodiment, the mutant type comprises a mutant form in which the function of the encoding protein is not altered after mutation (i.e., the function is the same or substantially the same as the wild-type encoding protein).

In another preferred embodiment, the mutant-type REST gene encodes a polypeptide that is the same or substantially the same as the polypeptide encoded by the wild-type REST gene.

In another preferred embodiment, the mutant-type REST gene comprises a sequence which is 80% or more (preferably 90% or more, more preferably 95% or more, much more preferably 98% or 99% or more) homology to the wild-type REST gene.

In another preferred embodiment, the mutant-type REST gene comprises a polynucleotide truncated or added with 1-60 (preferably 1-30, more preferably 1-10) nucleotides at the 5′- and/or 3′-end of the wild-type REST gene.

In another preferred embodiment, the REST gene comprises a cDNA sequence, a genomic sequence, or a combination thereof.

In another preferred embodiment, the REST protein comprises an active fragment of REST or a derivative thereof.

In another preferred embodiment, the active fragment or the derivative thereof has at least 90%, preferably 95%, more preferably 98% or 99% homology to the REST.

In another preferred embodiment, the active fragment or the derivative thereof has at least 80%, 85%, 90%, 95%, or 100% REST activity.

In another preferred embodiment, the amino acid sequence of the REST protein is selected from:
(i) a polypeptide having an amino acid sequence set forth in SEQ ID NO: 1;
(ii) a polypeptide formed by substituting, deleting or adding one or more (such as 1-10) amino acid residues in the amino acid sequence set forth above, having the protein function, and being derived from (i); or
(iii) a polypeptide having an amino acid sequence having 90% or more (preferably 95% or more, more preferably 98% or 99% or more) homology to the amino acid sequence set forth above and having the protein function.

In another preferred embodiment, the nucleotide sequence of the REST gene is selected from:
(a) a polynucleotide encoding a polypeptide having the amino acid sequence set forth above;
(b) a polynucleotide having a sequence set forth in SEQ ID NO: 2;
(c) a polynucleotide having a nucleotide sequence having 95% or more (preferably 98% or more, more preferably 99% or more) homology to the nucleotide sequence set forth above;
(d) a polynucleotide truncated or added with 1-60 (preferably 1-30, more preferably 1-10) nucleotides at the 5′- and/or 3′-end of a polynucleotide having the nucleotide sequence set forth above; and
a polynucleotide complementary to any one of the polynucleotides (a)-(d).

In another preferred embodiment, the REST protein is shown in the amino acid sequence described above.

In another preferred embodiment, the nucleic acid encoding the REST protein is shown in the nucleotide sequence described above.

In another preferred embodiment, the region targeted by the REST inhibitor (e.g., gene-editing protein) is positions 15311-15338 of the sequence of the REST gene.

In another preferred embodiment, the REST inhibitor inhibits the activity and/or the expression level of the REST.

In another preferred embodiment, the concentration of the REST inhibitor (titer of virus) is more than 1×10¹².

In another preferred embodiment, the inhibitory rate of the REST inhibitor against the activity and/or the expression level of the REST is more than 90%, preferably 90%-95%.

In another preferred embodiment, the inhibitor targets astrocytes of the brain tissue.

In another preferred embodiment, the inhibitor targets MG cells of the retina.

In another preferred embodiment, the gRNA guides the gene-editing protein to specifically bind to the mRNA of the REST gene.

In another preferred embodiment, the composition comprises a pharmaceutical composition.

In another preferred embodiment, the composition further comprises other drugs for preventing and/or treating a disease associated with loss of function or death of neurons.

In another preferred embodiment, the composition further comprises other drugs for treating a nervous system disease associated with death of functional neurons.

In another preferred embodiment, the composition further comprises other drugs for preventing and/or treating a retinal disease.

In another preferred embodiment, the expression vector of the gene-editing protein comprises a vector targeting glial cells.

In another preferred embodiment, the expression vector of the gene-editing protein comprises a vector targeting astrocytes of the brain tissue.

In another preferred embodiment, the expression vector of the gene-editing protein comprises a vector targeting MG cells of the retina.

In another preferred embodiment, the vector comprises AAV2, AAV8, or AAV9.

In another preferred embodiment, the gene encoding the gene-editing protein is located in the same expression vector (e.g., an AAV vector) as the gRNA.

In another preferred embodiment, the expression vector of the gene-editing protein and the expression vector of gRNA are the same expression vector (e.g., an AAV vector).

In another preferred embodiment, the expression vector further comprises a neuroglial cell-specific promoter (e.g., a GFAP promoter) for driving expression of the gene-editing protein.

In another preferred embodiment, the formulation of the composition is selected from: a lyophilized formulation, a liquid formulation, or a combination thereof.

In another preferred embodiment, the formulation of the composition is a liquid formulation.

In another preferred embodiment, the formulation of the composition is an injectable formulation.

In another preferred embodiment, the other drugs for preventing and/or treating a disease associated with loss of function or death of neurons are selected from: a dopamine prodrug, a non-ergot dopamine receptor agonist, a monoamine oxidase B inhibitor, or a combination thereof.

In another preferred embodiment, the composition is a cell formulation.

In another preferred embodiment, the expression vector of the gene-editing protein and the expression vector of the gRNA are the same vector or different vectors.

In another preferred embodiment, the weight ratio of the component (a) to the component (b) is 100:1-0.01:1, preferably 10:1-0.1:1, more preferably 2:1-0.5:1.

In another preferred embodiment, in the composition, the content of the component (a) is 0.001%-99%, preferably 0.1%-90%, more preferably 1%-70%.

In another preferred embodiment, in the composition, the content of the component (b) is 0.001%-99%, preferably 0.1%-90%, more preferably 1%-70%.

In another preferred embodiment, in the composition, the content of the component (c) is 1%-99%, preferably 10%-90%, more preferably 30%-70%.

In another preferred embodiment, in the composition, the component (a) and the component (b) and optionally the component (c) account for 0.01 wt %-99.99 wt %, preferably 0.1 wt %-90 wt %, more preferably 1 wt %-80 wt % of the total weight of the composition.

A third aspect of the present disclosure provides a package of drug, which comprises:
(a1) a first container, and a gene-editing protein or an expression vector thereof, or a drug comprising the gene-editing protein or the expression vector thereof, located in the first container, the gene-editing protein being selected from: Cas13d, CasRx, Cas13X, Cas13a, Cas13b, Cas13c, Cas13Y, an RNA-targeting gene-editing protein, or a combination thereof; and
(b1) a second container, and a gRNA or an expression vector thereof, or a drug comprising the gRNA or the expression vector thereof, located in the second container, the gRNA being a DNA or an RNA guiding the gene-editing protein to specifically bind to a REST gene.

In another preferred embodiment, the package of drug further comprises:
(c1) a third container, and other drugs for preventing and/or treating a disease associated with loss of function or death of neurons, located in the third container, and/or comprising other drugs for preventing and/or treating a retinal disease, and/or comprising other drugs for treating a nervous system disease associated with death of functional neurons.

In another preferred embodiment, the first container, the second container, and the third container are the same or different containers.

In another preferred embodiment, the drug in the first container is a single formulation comprising the gene-editing protein or the expression vector thereof.

In another preferred embodiment, the drug in the second container is a single formulation comprising the gRNA or the expression vector thereof.

In another preferred embodiment, the drug in the third container is a single formulation comprising other drugs for treating a nervous system disease associated with death of functional neurons.

In another preferred embodiment, the formulation of the drug is selected from: a lyophilized formulation, a liquid formulation, or a combination thereof.

In another preferred embodiment, the formulation of the drug is an oral formulation or an injectable formulation. In another preferred embodiment, the package further comprises an instruction.

It should be understood that within the scope of the present disclosure, the above various technical features of the present disclosure and those specifically described below (e.g., in the examples) may be combined with each other to form new or preferred technical solutions. Due to limited space, such schemes are not described herein.

### BRIEF DESCRIPTION OF THE DRAWINGS

FIG. 1. Analysis whether the glial cells can transdifferentiate into neurons in mice if miR124 is overexpressed. (A) is a schematic diagram of overexpression of miR 124 in the brain of a mouse. Vector-1 is used for labeling glial cells by using a glial cell-specific promoter GFAP to promote the expression of mCherry red fluorescent protein. Vector-2 is used for achieving specific expression of miR 124 in the glial cells by using the GFAP to promote the expression of miR124. (B) After injection of GFAP-mCherry+GFAP-miR124 into the striatum of a mouse, the orange arrows pointed to the labeled glial cells having morphological alterations, but not co-labeled with NeuN. (C) After injection of GFAP-mCherry+GFAP-miR124 into the striatum of a mouse, Tuj-1 being an early marker for neurons, the orange arrows pointed to the labeled glial cells which were not co-labeled with Tuj-1, with a scale of 40 microns. (D) After injection of GFAP-mCherry+GFAP-miR124 into the striatum of a mouse, staining was performed by using the neuron-specific marker NeuN and the dopamine neuron-specific marker TH, and the white arrows pointed to the labeled glial cells that were neither co-labeled with NeuN nor with the dopamine-specific marker TH, with a scale of 40 microns.

FIG. 2 shows screening of gRNA targeting mouse REST. (A) is a schematic diagram of plasmid construction. Vector-1 is a gRNA expression plasmid, gRNA was driven by U6, and meanwhile the red fluorescent protein was expressed to trace positively transfected cells: Vector-2 is a CasRx expression plasmid, CasRx was driven by CAG, and meanwhile the green fluorescent protein was expressed to trace positively transfected cells. (B) is a schematic diagram of cell transfection and fluorescence-activated cell sorting. After cell transfection, red positive cells and green positive cells were separated by using fluorescence-activated cell sorting, and the content of REST mRNA was measured by using QCPR. (C) After N2A cells were co-transferred with Cas13d and different gRNAs, positive cells were separated by using fluorescence-activated cell sorting, and the content of REST mRNA was measured by using QPCR after N2A cells were transfected with different gRNAs, a lower content of residual REST mRNA indicating higher knockdown efficiency of gRNAs. Red labeled gRNA-7 is most effective among the screened gRNAs.

FIG. 3 shows that the REST is inhibited to transdifferentiate glial cells into neurons in the brain of mice. (A) is a schematic diagram of vector construction and transdifferentiation of glial cells in the brain, where the labeling system is GFAP-mCherry, and expression of the fluorescent protein mCherry is promoted by an astrocyte-specific promoter GFAP: Vector 2 is an AAV plasmid in the control group, and CasRx expression is promoted by the astrocyte-specific promoter GFAP: Vector 3 is an AAV plasmid targeting REST, gRNA expression (corresponding to gRNA-7 of FIG. 2) is promoted by U6, and meanwhile CasRx expression is promoted by the astrocyte-specific promoter GFAP: different AAV combinations were injected into the brains of the mice, and materials were taken approximately 1 month after injection. (B) The virus (GFAP-mCherry+GFAP-CasRx) of the control group was injected into the striatum of the mice, orange arrows pointed to the labeled astrocytes, green for an astrocyte-specific marker GFAP, white for a mature neuron-specific marker NeuN, the nucleus was stained with Dapi, and the Merge images showed that mCherry signals were co-labeled with GFAP signals and not co-labeled with NeuN. (C) After co-injection of Vector 1 and Vector 3, the REST was knocked down in the striatum of the mice, glial cells were transdifferentiated into neurons, white for a mature neuron-specific marker NeuN, and white arrows pointed to the neurons co-labeled with mCherry and NeuN signals, with a scale of 20 microns. (D-E) After injection of the REST-knockdown AAV group (GFAP-mCherry+GFAP-CasRx-REST), a small fraction of fluorescently labeled cells in red expressed the dopamine neuron-specific cell markers TH and DAT.

FIG. 4 shows reduction of REST gene expression by using epigenetic regulation techniques. (A) is a schematic diagram of the epigenetic regulation principle, where DTM represents DNA targeting protein or protein structural domain (such as zinc finger protein, TALEs, CRISPR-dCas, etc.), DTM is connected with the epigenetic regulation protein and comprises DNA epigenetic modification related enzymes and histone modification related enzymes, and the expression of downstream gene is regulated under the action of DTM-epigenetic modifier. (B) is a schematic diagram of the plasmid vectors used in this study, with the U6 promoter driving expression of sgRNA and CMV driving expression of the red fluorescent protein (mCherry); in another vector, expression of the green fluorescent protein was promoted by an SV40 promoter, dCas9 (dSpCas9 or dSaCas9-KKH) was driven by EF1A for expression, and N2A cells were co-transferred with Vector 1 (U6-sgRNA-CMV-mCherry) and Vector 2 (dSpCas9-KRAB) or with Vector 1 and Vector 3 (dSaCas9-KKH-KRAB) for research and analysis. (C) After N2A cells were co-transformed with Vector 1 and Vector 2, the inhibitory effect of epigenetic regulation on the REST gene was detected by using Q-PCR. (D) After N2A cells were co-transformed with Vector 1 and Vector 3, the inhibitory effect of epigenetic regulation on the REST gene was detected by using Q-PCR.

FIG. 5 shows the screening of gRNAs in human cells (293T cells). (A) shows the knockdown efficiency of each gRNA against REST expression in 293T cells, red regions indicating gRNA regions with high knockdown efficiency. (B) is a REST expression line graph showing the REST knockdown condition of each gRNA, each gRNA corresponding to graph A. (C) shows the distribution position of each gRNA on the REST gene, red-labeled gRNAs being gRNAs with high inhibition efficiency, and magenta-labeled regions being efficient gRNA aggregation regions.

FIG. 6 shows efficient inhibition against the REST in different species. (A) 3 gRNA sequences targeting human REST and mismatched sites thereof in a cynomolgus monkey and a mouse are selected, bases labeled in red are sites in the cynomolgus monkey or mouse sequence that are different from those in the human REST sequence, and gRNA-17, gRNA-18 and gRNA-19 are gRNAs of the same serial number targeting human REST in FIG. 5. (B) is a schematic diagram of vector construction, where the gRNA in the expression vectors was driven by U6, the CasRx was driven by CAG, and a green fluorescent protein gene was added in the vector to label positively transfected cells. (C) is a schematic diagram of cell transfection and fluorescence-activated cell sorting. After transfection of different cells, EGFP positive cells were separated by using fluorescence-activated cell sorting and analyzed by using QPCR. (D) The expression level of REST mRNA was analyzed by using QPCR, and the gRNAs (gRNA-17, gRNA-18 and gRNA-19) targeting human REST can also efficiently knock down the expression level of mRNA in the REST of non-human primates (cynomolgus monkeys) and mice.

FIG. 7 shows that gRNAs targeting human REST can transdifferentiate glial cells into neurons. (A) is a schematic diagram of AAV vectors and the transdifferentiation process, where GFAP is an astrocyte-specific promoter, mCherry is a red fluorescent protein, CasRx is a gene-editing protein, U6-gRNA is a gRNA expression frame targeting REST promoted by U6, and the selected gRNA is gRNA-17 targeting human REST. Different combinations of AAV were injected into the striatum of mice, and the transdifferentiation effect was analyzed after 1 month. (B) The virus GFAP-mCherry+GFAP-CasRx in the control group was injected into the striatum of a mouse, the red fluorescence signal is GFAP-mCherry, the white fluorescence signal is a mature neuron-specific marker NeuN for staining, and mCherry and NeuN were not co-labeled with each other in the figure. (C) The virus combination of GFAP-mCherry+GFAP-CasRx-REST was injected into the striatum of a mouse, NeuN being a mature neuron-specific marker, and orange arrows pointed to neurons co-labeled with mCherry and NeuN, with a scale of 40 microns. (D) shows statistical analysis, showing the proportion of mCherry and NeuN double positive cells in mCherry positive cells (SEM, 3 mice per group). (E) The Müller glia were tried to be transdifferentiated into photoreceptor cells in the retinas, and GFAP is a promoter of the Müller glia in the retinas. After subretinal injection of virus GFAP-tdTomato+GFAP-CasRx-REST, wherein GFAP-tdTomato is used for labeling the retinal Müller glia, GFAP-CasRx-REST is used for knocking down REST in the Müller glia, and Rhodopsin is a specific protein marker of rod cells of photoreceptor cells in the retinas, cells indicated by white arrows simultaneously expressed tdTomato and Rhodopsin. (F) The Müller glia were tried to be transdifferentiated into retinal ganglion cells in the retina. After subretinal injection of virus GFAP-tdTomato+GFAP-CasRx-REST, red cells being GFAP-tdTomato-labeled cells, and green cells being cells stained with retinal ganglion cell-specific protein marker Rbpms, cells indicated by white arrows simultaneously expressed tdTomato and Rbpms, with a scale of 20 microns.

### DETAILED DESCRIPTION

The present inventor has made extensive and intensive studies and has found, for the first time, that inhibition of expression, content or activity of the gene of REST, an RNA thereof, or an encoding protein thereof in glial cells can effectively induce differentiation of glial cells into functional neurons, thereby treating nervous system diseases associated with loss of function or death of functional neurons. On the basis of this, the present inventor has completed the present invention.

In the present disclosure, degeneration of photoreceptor cells or retinal ganglion cells (RGCs) is the primary cause of permanent blindness. Transdifferentiation of Müller glia (MG) into functional photoreceptor cells or RGCs may help restore vision. The inventors found that by knocking down REST using the RNA-targeting CRISPR system CasRx in a mature mouse retina, MG cells can be directly transformed into functional photoreceptor cells or RGCs. Therefore, REST knockdown mediated by CasRx will be a promising therapy for the treatment of retinal diseases caused by neurodegeneration.

The present application uses the recently characterized RNA-targeting CRISPR system CasRx to inhibit REST. An excellent tool for treating various diseases is provided.

As used herein, Müller glia (MG) are the predominant neuroglial cells in retinal tissue. The retinal ganglion cells (RGCs) are nerve cells located in the innermost layers of the retina, their dendrites are mainly connected with bipolar cells, and their axons extend to the optic papilla to form optic nerve.

In the present disclosure, the gene editors comprise a DNA gene editor, an epigenetic regulatory editor, and an RNA gene editor. In a preferred embodiment, the gene editors of the present disclosure comprise gene-editing proteins and optionally gRNAs.

The term “reprogramming” or “transdifferentiation” may refer to the process of generating cells of a particular lineage (e.g., neurons) from different types of cells (e.g., astrocytes).

Diseases Associated with Loss of Function or Death of Neurons

In the present disclosure, diseases associated with loss of function or death of neurons mainly comprise diseases associated with loss of function or death of dopamine neurons, and visual impairment associated with loss of function or death of retinal ganglion cells or photoreceptor cells.

In a preferred embodiment, diseases associated with loss of function or death of neurons include, but are not limited to: Parkinson's disease, schizophrenia, depression, vision impairment due to death of RGCs, glaucoma, age-related RGC pathology, optic nerve damage, retinal ischemia or hemorrhage, Leber hereditary optic neuropathy, photoreceptor cell degeneration or death due to damage or degenerative diseases, macular degeneration, retinitis pigmentosa, diabetes-related blindness, night blindness, color blindness, inherited blindness, congenital amaurosis, etc.

### Astrocyte

Astrocytes are the most numerous cell type in the brain of mammals. They perform a number of functions, comprising biochemical support (e.g., forming a blood-brain barrier), providing nutrients for neurons, maintaining extracellular ionic balance, and participating in repair and scarring after brain and spinal cord injury. Astrocytes can be classified into two types according to the content of glial filaments and the shape of cytoplasmic processes: fibrous astrocytes mostly distributed in the white matter of the brain and spinal cord, having slender processes and fewer branches, and containing a large number of glial filaments in cytoplasm; and protoplasmic astrocytes mostly distributed in the gray matter, and having coarse and short cytoplasmic processes and many branches.

Astrocytes useful in the present disclosure are not particularly limited, and comprise various astrocytes derived from the mammalian central nervous system, for example, from the striatum, ventral tegmental area of the midbrain, hypothalamus, spinal cord, dorsal midbrain or cerebral cortex, preferably, from the striatum.

### Functional Neuron

In the present disclosure, functional neurons may refer to neurons capable of sending or receiving information by chemical or electrical signals. In some embodiments, functional neurons exhibit one or more functional properties of mature neurons present in the normal nervous system, including, but not limited to: excitability (e.g., the ability to exhibit an action potential, such as a rapid rise and subsequent fall) (voltage across cell membranes or membrane potential), formation of synaptic connections with other neurons, presynaptic neurotransmitter release, and postsynaptic responses (e.g., excitatory postsynaptic current or inhibitory postsynaptic current).

In some embodiments, the functional neurons are characterized by expressing one or more labels thereof, including, but not limited to, synaptoprotein, synapsin, glutamate decarboxylase 67 (GAD67), glutamate decarboxylase 65 (GAD65), parvalbumin, dopamine- and cAMP-regulated neuronal phosphoprotein 32 (DARPP32), vesicular glutamate transporter 1 (vGLUT1), vesicular glutamate transporter 2 (vGLUT2), acetylcholine, tyrosine hydroxylase (TH), dopamine, vesicular GABA transporter (VGAT), and γ-aminobutyric acid (GABA).

### Dopamine Neuron

Dopaminergic neurons contain and release dopamine (DA) as a neurotransmitter. Dopamine belongs to catecholamine neurotransmitters and plays an important biological role in the central nervous system. Dopaminergic neurons in the brain are mainly concentrated in the substantia nigra pars compacta (SNc) region of the midbrain, the ventral tegmental area (VTA), the hypothalamus, and around the brain ventricles. Many experiments have demonstrated that dopaminergic neurons are closely associated with a variety of diseases in the human body, most typically Parkinson's disease.

### Gene Editor

In the present disclosure, the gene editors comprise a DNA gene editor and an RNA gene editor. In a preferred embodiment, the gene editors of the present disclosure comprise gene-editing proteins and optionally gRNAs.

### Gene-Editing Protein

In the present disclosure, the nucleotide of the gene-editing protein can be obtained by genetic engineering techniques, such as genome sequencing, polymerase chain reaction (PCR), etc., and the amino acid sequence thereof can be deduced from the nucleotide sequence. Sources of the wild-type gene-editing proteins include (but are not limited to): Ruminococcus lavefaciens, Streptococcus pyogenes, Staphylococcus aureus, Acidaminococcus sp, Lachnospiraceae acterium.

In a preferred embodiment of the present disclosure, the gene-editing proteins include, but are not limited to, Cas13d, CasRx, Cas13X, Cas13a, Cas13b, Cas13c, Cas13Y, and RNA-targeting gene-editing proteins.

### REST Protein and Polynucleotide

In the present disclosure, the terms “proteins of the present disclosure”, “REST protein”, “REST polypeptide”, and “REST” are used interchangeably and all refer to a protein or polypeptide having a REST amino acid sequence. They comprise REST proteins with or without the initial methionine. In addition, the term also comprises full-length REST and fragments thereof. The REST proteins referred in the present disclosure comprise complete amino acid sequences thereof, secreted proteins thereof, mutants thereof, and functionally active fragments thereof.

REST proteins are repressor element 1-silencing transcription factors, also known as neuron-restrictive silencer factors (NRSFs).

In the present disclosure, the terms “REST gene”, “REST polynucleotide”, and “REST gene” are used interchangeably and all refer to a nucleic acid sequence having a REST nucleotide sequence.

The full length of the genome of the human REST gene is 27948 bp (NCBI GenBank accession number is 5978). The full length of the genome of the murine REST gene is 21007 bp (NCBI GenBank accession number is 19712).

Human and murine REST have 72% similarity at the DNA level and 62% protein sequence similarity. It should be understood that nucleotide substitutions in codons are acceptable when the same amino acids are encoded. It should be also understood that nucleotide changes may also be acceptable when conservative amino acid substitutions are made by nucleotide substitutions.

When an amino acid fragment of REST is obtained, a nucleic acid sequence encoding REST can be constructed therefrom, and a specific probe can be designed according to the nucleotide sequence. The full-length nucleotide sequence or a fragment thereof can be obtained by PCR amplification, recombination, or artificial synthesis. For PCR amplification, the primers can be designed according to the REST nucleotide sequences particularly the open reading frame sequences disclosed in the present disclosure, and the relevant sequences can be obtained by amplification using a commercially available cDNA library or a cDNA library prepared by a conventional method known to those skilled in the art as a template. When the sequence is long, it is often necessary to perform two or more PCR amplifications, and then the amplified fragments are spliced together in the correct order.

The relevant sequence, once obtained, can be replicated in large amount by recombination. This is implemented by cloning the sequence into a vector, transferring into a cell, and then isolating from proliferated host cells based on conventional methods.

In addition, the relevant sequence may be synthesized by artificial synthesis, especially when the fragment is short. Generally, a fragment with a long sequence can be obtained by first synthesizing multiple small fragments and then ligating them together.

A DNA sequence encoding the protein (or a fragment thereof, or a derivative thereof) of the present disclosure has already been obtained completely through chemical synthesis. The DNA sequence can then be introduced into various existing DNA molecules (such as vectors) and cells known in the art.

The polynucleotide sequences of the present disclosure can be used to express or produce recombinant REST polypeptides based on conventional recombinant DNA techniques. Generally, the following steps are provided:
(1). transforming or transducing a suitable host cell with a polynucleotide (or a variant) encoding a human REST polypeptide of the present disclosure, or with a recombinant expression vector comprising the polynucleotide;
(2). culturing the host cell in a suitable culture medium; and
(3). separating and purifying a protein from the culture medium or cells.

In the present disclosure, the REST polynucleotide sequence may be inserted into the recombinant expression vector. In general, any plasmid or vector can be used as long as it can replicate and is stable in the host. An important feature of expression vectors is that they typically comprise an origin of replication, a promoter, a marker gene, and translation control elements.

Methods well known to those skilled in the art can be used to construct expression vectors comprising the REST-encoding DNA sequence and appropriate transcriptional/translational control signals. These methods comprise in-vitro recombinant DNA techniques, DNA synthesis techniques, in-vivo recombinant techniques, etc. The DNA sequence may be operably linked to a suitable promoter in an expression vector to direct mRNA synthesis. The expression vector further comprises a ribosome binding site for translation initiation and a transcription terminator.

In addition, the expression vector preferably comprises one or more selectable marker genes to provide phenotypic traits for selection of transformed host cells, such as dihydrofolate reductase, neomycin resistance, and green fluorescent protein (GFP) for eukaryotic cell culture, or tetracycline or ampicillin resistance for Escherichia coli.

Vectors comprising the appropriate DNA sequences described above, and an appropriate promoter or a control sequence may be used to transform appropriate host cells to express protein.

The host cells may be prokaryotic cells, such as bacterial cells: or lower eukaryotic cells, such as yeast cells: or higher eukaryotic cells, such as mammalian cells. Representative examples comprise: Escherichia coli, Streptomyces bacterial cells: fungal cells such as yeast: plant cells: insect cells: animal cells, etc.

Transformation of host cells with recombinant DNA may be performed by conventional techniques well known to those skilled in the art. When the host is a prokaryote, such as Escherichia coli, competent cells capable of absorbing DNA can be harvested after exponential phase and processed by CaCl₂) transformation method according to steps that are well known in the art. Another method is to use MgCl₂. If necessary, the transformation can also be performed by electroporation. When the host is a eukaryote, the following DNA transfection methods can be used: calcium phosphate coprecipitation, conventional mechanical methods such as microinjection, electroporation, liposome packaging, and the like.

The obtained transformants can be cultivated by conventional methods to express the polypeptide encoded by the genes of the present disclosure. The medium is selected from various conventional media depending on the host cells used, and the host cells are incubated under conditions appropriate for their growth. After the host cells have been grown to an appropriate cell density, the selected promoter is induced by suitable methods (e.g., temperature conversion or chemical induction) and the cells are cultured for an additional period of time.

The recombinant polypeptide in the above method may be expressed intracellularly, or on the cell membrane, or secreted extracellularly. If necessary, the recombinant protein can be separated and purified by various isolation methods according to physical, chemical, and other properties. These methods are well known to those skilled in the art. Examples of these methods include, but are not limited to: conventional renaturation treatment, treatment with a protein precipitant (such as salt precipitation), centrifugation, osmotic lysis, sonication treatment, ultracentrifugation, molecular sieve chromatography (gel filtration), adsorption chromatography, ion exchange chromatography, high performance liquid chromatography (HPLC), and other various liquid chromatography techniques, and combinations thereof.

### Adeno-Associated Virus

Since adeno-associated viruses (AAVs) are smaller than other viral vectors, are not pathogenic, and can transfect dividing and non-dividing cells, gene therapy methods based on AAV vectors for genetic diseases have received much attention.

Adeno-associated viruses, also known as AAVs, belong to the genus Dependoparvovirus of the family Parvoviridae, and are the simplest single-stranded DNA-defective virus of the group of viruses currently discovered, requiring a helper virus (usually adenovirus) to participate in replication. It encodes the cap and rep genes in two inverted terminal repeats (ITRs). ITRs are crucial for replication and packaging of viruses. The cap gene encodes the capsid protein of the virus, and the rep gene is involved in the replication and integration of the virus. AAVs can infect a variety of cells.

The recombinant adeno-associated viral (rAAV) vector is derived from non-pathogenic wild-type adeno-associated virus, is considered to be one of the most promising gene transfer vectors due to the characteristics of good safety, wide host cell range (dividing and non-dividing cells), low immunogenicity, long duration for expressing exogenous genes in vivo and the like, and is widely applied to gene therapy and vaccine research in the world. Over 10 years of research, the biological properties of recombinant adeno-associated viruses have been well understood, with a lot of data having been accumulated especially in the aspect of their application effect in various cells, tissues and in-vivo experiments. In medical research, rAAVs are used in the study of gene therapy for a variety of diseases (comprising in-vivo experiments, and in-vitro experiments); meanwhile, rAAVs, as characteristic gene transfer vectors, are widely applied to the aspects of gene function research, disease model construction, gene knock-out mouse preparation, and the like.

In a preferred embodiment of the present disclosure, the vector is a recombinant AAV vector. AAVs are relatively small DNA viruses that can be integrated into the genome of cells that they infect in a stable and site-specific manner. They can infect a large series of cells without any effect on cell growth, morphology or differentiation, and they do not appear to be involved in human pathology. AAV genomes have been cloned, sequenced, and characterized. An AAV comprises an inverted terminal repeat (ITR) region of about 145 bases at each terminus, which serves as the viral origin of replication. The remainder of the genome is divided into two important regions with encapsidation functions: the left part of the genome comprising the rep gene involved in viral replication and viral gene expression; and the right part of the genome comprising the cap gene encoding the viral capsid protein.

AAV vectors can be prepared using standard methods in the art. Any serotype of adeno-associated virus is suitable. Methods for purifying vectors can be found, for example, in U.S. Pat. Nos. 6,566,118, 6,989,264, and 6,995,006, the disclosures of which are incorporated herein by reference in their entireties. Preparation of hybrid vectors is described, for example, in PCT application No. PCT/US2005/027091, the disclosure of which is incorporated herein by reference in its entirety. The use of vectors derived from AAVs for in-vitro and in-vivo transfer genes has been described (see, e.g., International Patent Application Publication Nos. WO91/18088 and WO 93/09239; U.S. Pat. Nos. 4,797,368, 6,596,535, and 5,139,941, and European Pat. No. 0488528, all of which are incorporated herein by reference in their entireties). These patent publications describe various AAV-derived constructs in which the rep and/or cap genes are deleted and replaced by a gene of interest, and the use of these constructs to transport the gene of interest in vitro (into cultured cells) or in vivo (directly into an organism). Recombinant replication-deficient AAVs can be prepared by co-transfecting the following plasmids into cell lines infected with a human helper virus (e.g., adenovirus): plasmids containing the nucleic acid sequence of interest flanked by two regions of AAV inverted terminal repeats (ITRs), and plasmids carrying AAV encapsidation genes (rep and cap genes). The AAV recombinants produced are then purified by standard techniques.

In some embodiments, the recombinant vectors are encapsidated into virions (e.g., AAV virions including, but not limited to, AAV1, AAV2, AAV3, AAV4, AAV5, AAV6, AAV7, AAV8, AAV9, AAV-DJ, AAV-rh10, PHP.S, PHP.B, PHP.eB, and AAV2-7m8). Accordingly, the present disclosure comprises recombinant virions (recombinant in that they comprise a recombinant polynucleotide) comprising any of the vectors described herein. Methods for producing such particles are known in the art and are described in U.S. Pat. No. 6,596,535.

### Rest Inhibitor and Pharmaceutical Composition

Substances, particularly inhibitors and the like, which interact with the REST gene or protein, can be picked out by various conventional screening methods using the proteins of the present disclosure.

REST inhibitors (or antagonists) useful in the present disclosure may be substances that reduce and eliminate the expression, content and/or activity of the gene of REST, an RNA (e.g., mRNA) thereof, or an encoding protein thereof at the DNA, RNA, protein level.

For example, the REST inhibitors comprise an antibody against REST, an antisense RNA against REST nucleic acid, siRNA, shRNA, miRNA, a gene editor, or a REST activity inhibitor. A preferred REST inhibitor refers to a gene editor capable of inhibiting REST expression.

Preferably, the REST inhibitors of the present disclosure comprise inhibitors targeting positions 15311-15338 of the REST gene sequence. The subjects on which the REST inhibitors of the present disclosure act comprise astrocytes or MG cells.

In a preferred embodiment, the method and steps for inhibiting REST comprise using an antibody against REST to neutralize REST proteins, using shRNA or siRNA or a gene editor carried by a virus (e.g., adeno-associated virus) to silence the REST gene.

The inhibition rate of the REST is generally at least 50% or more, preferably at least 60%, 70%, 80%, 90%, 95% or more, and can be controlled and detected based on conventional techniques, such as flow cytometry, fluorescence quantitative PCR or Western blot, etc.

The REST inhibitors (comprising antibodies, antisense nucleic acids, gene editors, and other inhibitors) of the present disclosure, when administered (dosed) therapeutically, can inhibit the expression and/or activity of REST proteins, thereby inducing differentiation of glial cells into functional neurons, thus treating diseases associated with loss of function or death of neurons. Generally, these materials can be formulated in a non-toxic, inert and pharmaceutically acceptable aqueous carrier medium, wherein the pH is typically about 5-8, preferably about 6-8, although the pH may vary depending on the properties of the material being formulated and the condition being treated. The formulated pharmaceutical composition may be administered by conventional routes including, but not limited to: locally, intramuscular, intracranial, intraocular, intraperitoneal, intravenous, subcutaneous, intradermal administration, autologous cell extraction culture followed by reinfusion, etc.

The present disclosure further provides a pharmaceutical composition, which comprises a safe and effective amount of the inhibitor of the present disclosure (e.g., an antibody, a gene editor, an antisense sequence (e.g., siRNA), or an inhibitor), and a pharmaceutically acceptable carrier or excipient. Such vectors include (but are not limited to): saline, buffer, glucose, water, glycerol, ethanol, and combinations thereof. The pharmaceutical formulation shall match the route of administration. The pharmaceutical composition of the present disclosure may be prepared in the form of injections, for example, using normal saline or an aqueous solution containing glucose and other adjuvants, by a conventional method. The pharmaceutical compositions in the form of a tablet and a capsule may be prepared by a conventional method. The pharmaceutical compositions in the form of an injection, a solution, a tablet and a capsule are preferably manufactured under sterile conditions. The amount of the active ingredient administered is a therapeutically effective amount, for example, from about 1 μg/kg body weight to about 10 mg/kg body weight per day.

The main advantages of the present disclosure comprise that:
(1) The present disclosure has found for the first time that reduction of the expression, content or activity of a gene of REST or an encoding protein thereof in astrocytes can induce differentiation of astrocytes into dopamine neurons, thereby preventing and/or treating Parkinson's disease.
(2) The present disclosure has found for the first time that the inhibition against expression of REST in astrocytes by using gene editors (comprising gene-editing proteins and gRNAs) can transdifferentiate the astrocytes into dopamine neurons, thereby providing a potential approach to the treatment of Parkinson's disease.
(3) The present disclosure has found for the first time that an RNA-targeting CRISPR system CasRx can avoid the risk of permanent DNA changes caused by traditional CRISPR-Cas9 editing. Therefore, CasRx-mediated RNA editing provides an effective means for treating various diseases.
(4) The present disclosure converts MG cells directly into functional photoreceptor cells and RGCs by inhibiting the expression of REST in the retina.
(5) The present disclosure uses the RNA-targeting CRISPR system CasRx to knock down REST, providing an excellent tool capable of treating a variety of diseases.

The present disclosure is further illustrated with reference to the following specific examples. It should be understood that these examples are for illustrative purposes only and are not intended to limit the scope of the present disclosure. Experimental procedures without specific conditions indicated in the following examples are generally performed based on conventional conditions, such as conditions described in Sambrook et al., Molecular Cloning: Laboratory Manual (New York: Cold Spring Harbor Laboratory Press, 1989), or conditions recommended by the manufacturers. Unless otherwise stated, percentages and parts are by weight.

Unless otherwise indicated, all materials and reagents used in the examples of the present disclosure are commercially available products.

### General Method

Ethics of animals: the use and the feeding of the animals accord with the guiding principle of the Ethical Committee of the Biomedical Research of the Center for Excellence in Brain Science and Intelligence Technology.

### Guide RNA Sequence

The guide RNA targeting mouse REST is, for example, set forth in SEQ ID NOs: 4-20, preferably SEQ ID NO: 10.

Transient Transfection and qPCR Analysis of Cos7 Cells, 293T or N2a Cells

Cell lines were transiently transfected with 4 μg of CAG-CasRx-P2A-GFP plasmid and 2 μg of U6-gRNA-CMV-mCherry plasmid to determine the inhibitory effect on REST in cell lines in vitro. Meanwhile, the CAG-CasRx-P2A-GFP plasmid was used as a control group for single transfection. Lipofectamine 3000 (Thermo Fisher Scientific) was used according to standard procedures. Two days after transfection, 30000 GFP and mCherry double positive cells were harvested for each sample by fluorescence-activated cell sorting (FACS) (EGFP positive cells were harvested for the control group). The harvested cells were RNA-extracted with Trizol (Ambion) and reversely transcribed into cDNA using a reverse transcription kit (HiScript Q RT SuperMix for qPCR, Vazyme, Biotech), and QPCR analysis was performed using AceQ qPCR SYBR Green Master Mix (Vazyme, Biotech).

The mouse REST-targeting qPCR primers were as follows: an upstream primer, 5′-ctggctcttccactgcagaa-3′ (SEQ ID NO: 193); a downstream primer, 5′-tggtgtttcaggtgtgctgt-3′ (SEQ ID NO: 194);
The mouse GAPDH-targeting qPCR primers were as follows: an upstream primer, 5′-ctacccccaatgtgtccgtc-3′ (SEQ ID NO: 195); a downstream primer, 5′-aagtcgcaggagacaacctg-3′ (SEQ ID NO: 196);
The human and cynomolgus monkey REST-targeting qPCR primers were as follows: an upstream primer, 5′-gttagaactcatacaggaga-3′ (SEQ ID NO: 197): a downstream primer, 5′-gaggtttaggcccattgtga-3′ (SEQ ID NO: 198);
The human GAPDH-targeting qPCR primers were as follows: an upstream primer, 5′-gtctcctctgacttcaacagcg-3′ (SEQ ID NO: 199); a downstream primer, 5′-accaccctgttgctgtagccaa-3′ (SEQ ID NO: 200);
The cynomolgus monkey GAPDH-targeting qPCR primers were as follows: an upstream primer, 5′-ggtcaccagggctgctttta-3′ (SEQ ID NO: 201); a downstream primer, 5′-ttcccgttetcagccttcac-3′ (SEQ ID NO: 202).

### Stereotactic Injection

The AAV serotype used in this study was AAV8, and the method for stereotactic injection (C57BL/6, approximately two months old) was as described above 2. An AAV mixed solution with the titer of greater than 5×10¹² vg/mL was injected into the striatum (AP+0.8 mm, ML+1.6 mm, and DV-2.8 mm) using a stereotactic injector at an amount of 1 μL. In miR124 overexpression experiment, the AAV injected was AAV-GFAP-miR124 (approximately 1.7×10¹³ vg/mL). In the REST knockdown experiment, viruses in the control group were GFAP-mCherry (approximately 5×10¹¹ vg/mL)+AAV-GFAP-CasRx (titer was approximately 1.2×10¹³ vg/mL, with no gRNAs targeting REST), and AAV viruses in the experimental group were GFAP-mCherry+AAV-GFAP-CasRx-REST (titer was approximately 1.2×10¹³ vg/mL, comprising gRNAs targeting REST), and 1-3 mice were injected per group.

### Subretinal Injection

AAV8 was injected subretinally as described above. For subretinal injection, an AAV was injected subretinally using an Olympus microscope (Olympus, Japan) using a Hamilton syringe (32G needle). To determine reprogramming in the intact retinas, a total of 1 μL of GFAP-tdTomato (0.1 μL, approximately 1×10¹² vg/mL) and GFAP-CasRx-REST (0.9 μL, approximately 1.2×10¹³ vg/mL), or GFAP-tdTomato (0.1 μL, approximately 1×10¹² vg/mL) and GFAP-CasRx (0.9 μL, approximately 1.2×10¹³ vg/mL) was injected subretinally (C57BL/6 mice, approximately 5 weeks old).

### Immunofluorescence Staining

Immunofluorescence staining for brain: approximately 1 month after injection, the mice were perfused to remove the brains, and the brains were fixed with 4% paraformaldehyde (PFA) overnight and dehydrated in 30% sucrose for at least 12 hours. The brains were embedded and made into frozen sections with a thickness of 30 μm. Brain sections were thoroughly rinsed with 0.1 M phosphate buffer (PB) prior to immunofluorescence staining. Primary antibodies for immunofluorescence staining were as follows: rabbit polyclonal NeuN antibody (1:500, #ABN78, Millipore), mouse TH antibody (1:300, MAB318, Millipore), and rat DAT antibody (1:100, MAB369, Millipore). The secondary antibodies were as follows: Alexa Fluor® 488 AffiniPure Donkey Anti-Mouse IgG (H+L) (1:500, 715-545-150, Jackson ImmunoResearch), Alexa Fluor® 488 AffiniPure Donkey Anti-Rabbit IgG (H+L) (1:500, 711-545-152, Jackson ImmunoResearch); Alexa Fluor® 488 AffiniPure Donkey Anti-Rat IgG (H+L) (1:500, 712-545-153, Jackson ImmunoResearch); Cy™5 AffiniPure Donkey Anti-Rabbit IgG (H+L) (1:500, 711-175-152, Jackson ImmunoResearch). After antibody incubation, sections were washed and mounted with a mounting medium (Life Technology). For retinal sections, approximately 1 month after AAV injection, eyes were taken, fixed with 4% paraformaldehyde (PFA) for 2 hours (eyes), and dehydrated in 30% sucrose solution, and then the tissue was sectioned in an embedding cassette with a thickness of 30 μm. Primary antibodies for immunofluorescence staining were as follows: rabbit anti-RBPMS (1:500, 15187-1-AP, Proteintech), mouse-anti-rhodopsin (1:2000, MAB5356, EMD Millipore) and secondary antibodies were as follows: Alexa Fluor® 488 AffiniPure Donkey Anti-Rabbit IgG (H+L) (1:500, 715-545-150, Jackson ImmunoResearch), Alexa Fluor® 488 AffiniPure Donkey Anti-Mouse IgG (H+L) (1:500, 711-545-152, Jackson ImmunoResearch). After antibody incubation, the sections were washed and mounted. Imaging was performed using an Olympus FV3000 microscope.

### Sequence Information

### Amino Acid Sequence of Human REST Protein (SEQ ID NO: 1):

MATQVMGQSSGGGGLFTSSGNIGMALPNDMYDLHDLSKAELAAPQL
IMLANVALTGEVNGSCCDYLVGEERQMAELMPVGDNNFSDSEEGE
GLEESADIKGEPHGLENMELRSLELSVVEPQPVFEASGAPDIYSS
NKDLPPETPGAEDKGKSSKTKPFRCKPCQYEAESEEQFVHHIRVH
SAKKFFVEESAEKQAKARESGSSTAEEGDFSKGPIRCDRCGYNTN
RYDHYTAHLKHHTRAGDNERVYKCIICTYTTVSEYHWRKHLRNHF
PRKVYTCGKCNYFSDRKNNYVQHVRTHTGERPYKCELCPYSSSQK
THLTRHMRTHSGEKPFKCDQCSYVASNQHEVTRHARQVHNGPKPL
NCPHCDYKTADRSNFKKHVELHVNPRQFNCPVCDYAASKKCNLQY
HFKSKHPTCPNKTMDVSKVKLKKTKKREADLPDNITNEKTEIEQT
KIKGDVAGKKNEKSVKAEKRDVSKEKKPSNNVSVIQVTTRTRKSV
TEVKEMDVHTGSNSEKFSKTKKSKRKLEVDSHSLHGPVNDEESST
KKKKKVESKSKNNSQEVPKGDSKVEENKKQNTCMKKSTKKKTLKN
KSSKKSSKPPQKEPVEKGSAQMDPPQMGPAPTEAVQKGPVQVEPP
PPMEHAQMEGAQIRPAPDEPVQMEVVQEGPAQKELLPPVEPAQMV
GAQIVLAHMELPPPMETAQTEVAQMGPAPMEPAQMEVAQVESAPM
QVVQKEPVQMELSPPMEVVQKEPVQIELSPPMEVVQKEPVKIELS
PPIEVVQKEPVQMELSPPMGVVQKEPAQREPPPPREPPLHMEPIS
KKPPLRKDKKEKSNMQSERARKEQVLIEVGLVPVKDSWLLKESVS
TEDLSPPSPPLPKENLREEASGDQKLLNTGEGNKEAPLQKVGAEE
ADESLPGLAANINESTHISSSGQNLNTPEGETLNGKHQTDSIVCE
MKMDTDQNTRENLTGINSTVEEPVSPMLPPSAVEEREAVSKTALA
SPPATMAANESQEIDEDEGIHSHEGSDLSDNMSEGSDDSGLHGAR
PVPQESSRKNAKEALAVKAAKGDFVCIFCDRSFRKGKDYSKHLNR
HLVNVYYLEEAAQGQE

### Human REST Coding Sequence (SEQ ID NO: 2):

atggccacccaggtaatggggcagtcttctggaggaggagggctg
tttaccagcagtggcaacattggaatggccctgcctaacgacatg
tatgacttgcatgacctttccaaagctgaactggccgcacctcag
cttattatgctggcaaatgtggccttaactggggaagtaaatggc
agctgctgtgattacctggtcggtgaagaaagacagatggcagaa
ctgatgccggttggggataacaacttttcagatagtgaagaagga
gaaggacttgaagagtctgctgatataaaaggtgaacctcatgga
ctggaaaacatggaactgagaagtttggaactcagcgtcgtagaa
cctcagcctgtatttgaggcatcaggtgctccagatatttacagt
tcaaataaagatcttccccctgaaacacctggagcggaggacaaa
ggcaagagctcgaagaccaaaccctttcgctgtaagccatgccaa
tatgaagcagaatctgaagaacagtttgtgcatcacatcagagtt
cacagtgctaagaaattttttgtggaagagagtgcagagaagcag
gcaaaagccagggaatctggctcttccactgcagaagagggagat
ttctccaagggccccattcgctgtgaccgctgcggctacaatact
aatcgatatgatcactatacagcacacctgaaacaccacaccaga
gctggggataatgagcgagtctacaagtgtatcatttgcacatac
acaacagtgagcgagtatcactggaggaaacatttaagaaaccat
tttccaaggaaagtatacacatgtggaaaatgcaactatttttca
gacagaaaaaacaattatgttcagcatgttagaactcatacagga
gaacgcccatataaatgtgaactttgtccttactcaagttctcag
aagactcatctaactagacatatgcgtactcattcaggtgagaag
ccatttaaatgtgatcagtgcagttatgtggcctctaatcaacat
gaagtaacccgccatgcaagacaggttcacaatgggcctaaacct
cttaattgcccacactgtgattacaaaacagcagatagaagcaac
ttcaaaaaacatgtagagctacatgtgaacccacggcagttcaat
tgccctgtatgtgactatgcagcttccaagaagtgtaatctacag
tatcacttcaaatctaagcatcctacttgtcctaataaaacaatg
gatgtctcaaaagtgaaactaaagaaaaccaaaaaacgagaggct
gacttgcctgataatattaccaatgaaaaaacagaaatagaacaa
acaaaaataaaaggggatgtggctggaaagaaaaatgaaaagtcc
gtcaaagcagagaaaagagatgtctcaaaagagaaaaagccttct
aataatgtgtcagtgatccaggtgactaccagaactcgaaaatca
gtaacagaggtgaaagagatggatgtgcatacaggaagcaattca
gaaaaattcagtaaaactaagaaaagcaaaaggaagctggaagtt
gacagccattctttacatggtcctgtgaatgatgaggaatcttca
acaaaaaagaaaaagaaggtagaaagcaaatccaaaaataatagt
caggaagtgccaaagggtgacagcaaagtggaggagaataaaaag
caaaatacttgcatgaaaaaaagtacaaagaagaaaactctgaaa
aataaatcaagtaagaaaagcagtaagcctcctcagaaggaacct
gttgagaagggatctgctcagatggaccctcctcagatggggcct
gctcccacagaggcggttcagaaggggcccgttcaggtggagccg
ccacctcccatggagcatgctcagatggagggtgcccagatacgg
cctgctcctgacgagcctgttcagatggaggtggttcaggagggg
cctgctcagaaggagctgctgcctcccgtggagcctgctcagatg
gtgggtgcccaaattgtacttgctcacatggagctgcctcctccc
atggagactgctcagacggaggttgcccaaatggggcctgctccc
atggaacctgctcagatggaggttgcccaggtagaatctgctccc
atgcaggtggtccagaaggagcctgttcagatggagctgtctcct
cccatggaggtggtccagaaggagcctgttcagatagagctgtct
cctcccatggaggtggtccagaaggaacctgttaagatagagctg
tctcctcccatagaggtggtccagaaggagcctgttcagatggag
ttgtctcctcccatgggggtggttcagaaggagcctgctcagagg
gagccacctcctcccagagagcctccccttcacatggagccaatt
tccaaaaagcctcctctccgaaaagataaaaaggaaaagtctaac
atgcagagtgaaagggcacggaaggagcaagtccttattgaagtt
ggcttagtgcctgttaaagatagctggcttctaaaggaaagtgta
agcacagaggatctctcaccaccatcaccaccactgccaaaggaa
aatttaagagaagaggcatcaggagaccaaaaattactcaacaca
ggtgaaggaaataaagaagcccctcttcagaaagtaggagcagaa
gaggcagatgagagcctacctggtcttgctgctaatatcaacgaa
tctacccatatttcatcctctggacaaaacttgaatacgccagag
ggtgaaactttaaatggtaaacatcagactgacagtatagtttgt
gaaatgaaaatggacactgatcagaacacaagagagaatctcact
ggtataaattcaacagttgaagaaccagtttcaccaatgcttccc
ccttcagcagtagaagaacgtgaagcagtgtccaaaactgcactg
gcatcacctcctgctacaatggcagcaaatgagtctcaggaaatt
gatgaagatgaaggcatccacagccatgaaggaagtgacctaagt
gacaacatgtcagagggtagtgatgattctggattgcatggggct
cggccagttccacaagaatctagcagaaaaaatgcaaaggaagcc
ttggcagtcaaagcggctaagggagattttgtttgtatcttctgt
gatcgttctttcagaaagggaaaagattacagcaaacacctcaat
cgccatttggttaatgtgtactatcttgaagaagcagctcaaggg
caggagtaa

### Nucleotide Sequence at Positions 867-1103 of Human REST Coding Sequence (SEQ ID NO: 3):

caattatgttcagcatgttagaactcatacaggagaacgcccata
taaatgtgaactttgtccttactcaagttctcagaagactcatct
aactagacatatgcgtactcattcaggtgagaagccatttaaatg
tgatcagtgcagttatgtggcctctaatcaacatgaagtaacccg
ccatgcaagacaggttcacaatgggcctaaacctcttaattgccc
acactgtgatta

gRNAs Efficiently Targeting REST Nicked Out in Mouse NA2 Cells (SEO ID NOs: 4-54)

| | SEQ ID | | SEQ ID | Target coding | SEQ ID | Target mRNA |
|---|---|---|---|---|---|---|
| gRNA | NO. | gRNA sequence | NO. | sequence | NO. | sequence |
| mouse- | 4 | agcucgugcaggucg | 21 | aaccaacgacatgta | 38 | aaccaacgacaugua |
| REST- | | uacaugucguugguu | | cgacctgcacgagct | | cgaccugcacgagcu |
| gRNA1 | | | | | | |
| mouse- | 5 | cgcuguauauuucug | 22 | gaagcctcagctgcc | 39 | gaagccucagcugcc |
| REST- | | ggcagcugaggcuuc | | ccagaaatatacagc | | ccagaaauauacagc |
| gRNA2 | | | | g | | g |
| mouse- | 6 | cuuuggccuguuucu | 23 | gtggaggaaagtgca | 40 | guggaggaaagugca |
| REST- | | cugcacuuuccucca | | gagaaacaggccaaa | | gagaaacaggccaaa |
| gRNA3 | | c | | g | | g |
| mouse- | 7 | cacuugcugcaggug | 24 | ccccaggaaagtcta | 41 | ccccaggaaagucua |
| REST- | | uagacuuuccugggg | | cacctgcagcaagtg | | caccugcagcaagug |
| gRNA4 | | | | | | |
| mouse- | 8 | gcguucuccugugug | 25 | cagcacgtgcgaact | 42 | cagcacgugcgaacu |
| REST- | | aguucgcacgugcug | | cacacaggagaacgc | | cacacaggagaacgc |
| gRNA5 | | | | | | |
| mouse- | 9 | gaugagucuucugag | 26 | gtccttactcaagct | 43 | guccuuacucaagcu |
| REST- | | agcuugaguaaggac | | ctcagaagactcatc | | cucagaagacucauc |
| gRNA6 | | | | | | |
| mouse- | 10 | gucacuucaugcuga | 27 | atgtggcctctaatc | 44 | auguggccucuaauc |
| REST- | | uuagaggccacau | | agcatgaagtgac | | agcaugaagugac |
| gRNA7 | | | | | | |
| mouse- | 11 | cgggcaauuaagagg | 28 | cacaacgggcctaaa | 45 | cacaacgggccuaaa |
| REST- | | uuuaggcccguugug | | cctcttaattgcccg | | ccucuuaauugcccg |
| gRNA8 | | | | | | |
| mouse- | 12 | ucacacacggggcag | 29 | aacccacggcagttc | 46 | aacccacggcaguuc |
| REST- | | uugaacugccguggg | | aactgccccgtgtgt | | aacugccccgugugu |
| gRNA9 | | uu | | ga | | ga |
| mouse- | 13 | ugguauuguagauua | 30 | ctaagaagtgtaatc | 47 | cuaagaaguguaauc |
| REST- | | cacuucuuag | | tacaatacca | | uacaauacca |
| gRNA10 | | | | | | |
| mouse- | 14 | cugggacaggugggau | 31 | caaatctaagcatcc | 48 | caaaucuaagcaucc |
| REST- | | gcuuagauuug | | cacctgtcccag | | caccugucccag |
| gRNA11 | | | | | | |
| mouse- | 15 | ucuucucguugcuga | 32 | aataacgccgtcagc | 49 | aauaacgccgucagc |
| REST- | | cggcguuauu | | aacgagaaga | | aacgagaaga |
| gRNA12 | | | | | | |
| mouse- | 16 | gcggcgucguucuuu | 33 | cccttaagaaaggca | 50 | cccuuaagaaaggca |
| REST- | | gugccuuucuuaagg | | caaagaagacgccgc | | caaagaagacgccgc |
| gRNA13 | | g | | | | |
| mouse- | 17 | agaagauccugaccc | 34 | caggcagaggtcacc | 51 | caggcagaggucacc |
| REST- | | ggugaccucugccug | | gggtcaggatcttct | | gggucaggaucuucu |
| gRNA14 | | | | | | |
| mouse- | 18 | gcuccauacugggag | 35 | cccagaaggaaccac | 52 | cccagaaggaaccac |
| REST- | | gugguuccuucuggg | | ctcccagtatggagc | | cucccaguauggagc |
| gRNA15 | | | | | | |
| mouse- | 19 | aagcuugcugucucu | 36 | ggcttggtgcctgtt | 53 | ggcuuggugccuguu |
| REST- | | aacaggcaccaagcc | | agagacagcaagctt | | agagacagcaagcuu |
| gRNA16 | | | | | | |
| mouse- | 20 | aucugucuucugcuc | 37 | gtgacgtggacactg | 54 | gugacguggacacug |
| REST- | | aguguccacgucac | | agcagaagacagat | | agcagaagacagau |
| gRNA17 | | | | | | |

sgRNA Guide Sequences for Epigenetic Methods (SEQ ID NOs: 55-82)
sgRNA1-8 of dSpCas9-KRAB

| | SEQ | | SEQ | |
|---|---|---|---|---|
| | ID | | ID | |
| gRNA | NO. | Guide sequence | NO. | DNA target |
| sgRNA1 | 55 | ggcgcagcag | 63 | ggcgcagcag |
| | | cagaagaccg | | cagaagaccg |
| sgRNA2 | 56 | accgcagcga | 64 | accgcagcga |
| | | cggcagaacc | | cggcagaacc |
| sgRNA3 | 57 | ccctggttct | 65 | ccctggttct |
| | | gccgtcgctg | | gccgtcgctg |
| sgRNA4 | 58 | agcgacggca | 66 | agcgacggca |
| | | gaaccagggc | | gaaccagggc |
| sgRNA5 | 59 | cgggatcaga | 67 | cgggatcaga |
| | | ccgccggccc | | ccgccggccc |
| sgRNA6 | 60 | gatcgcaccc | 68 | gatcgcaccc |
| | | cgggatctcg | | cgggatctcg |
| sgRNA7 | 61 | gagttggagc | 69 | gagttggagc |
| | | ggcggcgacg | | ggcggcgacg |
| sgRNA8 | 62 | atactgtggc | 70 | atactgtggc |
| | | tcgggcggcg | | tcgggcggcg |

Sgrna1-6 of dSaCas9-KKH-Krab

| | SEQ | | SEQ | |
|---|---|---|---|---|
| | ID | Guide | ID | |
| gRNA | NO. | sequence | NO. | DNA target |
| sgRNA | 71 | ggcgggcggcg | 77 | ggcgggcggcg |
| 1 | | acggcgcggg | | acggcgcggg |
| sgRNA | 72 | gcgcggcgcag | 78 | gcgcggcgcag |
| 2 | | cgtcctgtgc | | cgtcctgtgc |
| sgRNA | 73 | agcgacggcag | 79 | agcgacggcag |
| 3 | | aaccagggcc | | aaccagggcc |
| sgRNA | 74 | cggccctggtt | 80 | cggccctggtt |
| 4 | | ctgccgtcgc | | ctgccgtcgc |
| sgRNA | 75 | ggaccgtgggc | 81 | ggaccgtgggc |
| 5 | | gcacagttca | | gcacagttca |
| sgRNA | 76 | cggccgccgcg | 82 | cggccgccgcg |
| 6 | | ccgcccgagc | | ccgcccgagc |

A Series of gRNA Targeting Human REST mRNA Constructed (SEQ ID NOs: 83-190)

| | SEQ | | SEQ | | SEQ | |
|---|---|---|---|---|---|---|
| | ID | | ID | Target coding | ID | |
| gRNA | NO. | gRNA sequence | NO. | sequence | NO. | Target mRNA sequence |
| human | 83 | uaagcugaggugcgg | 119 | ccaaagctgaactgg | 155 | ccaaagcugaacugg |
| REST- | | ccaguucagcuuugg | | ccgcacctcagctta | | ccgcaccucagcuua |
| gRN | | | | | | |
| A1 | | | | | | |
| human | 84 | ucacagcagcugcca | 120 | aactggggaagtaaa | 156 | aacuggggaaguaaa |
| REST- | | uuuacuuccccaguu | | tggcagctgctgtga | | uggcagcugcuguga |
| gRN | | | | | | |
| A2 | | | | | | |
| human | 85 | ccaucugucuuucuu | 121 | acctggtcggtgaag | 157 | accuggucggugaag |
| REST- | | caccgaccaggu | | aaagacagatgg | | aaagacagaugg |
| gRN | | | | | | |
| A3 | | | | | | |
| human | 86 | cuguaaauaucugga | 122 | gaggcatcaggtgct | 158 | gaggcaucaggugcu |
| REST- | | gcaccugaugccuc | | ccagatatttacag | | ccagauauuuacag |
| gRN | | | | | | |
| A4 | | | | | | |
| human | 87 | uucugcuucauaccg | 123 | cgctgtaagccatgc | 159 | cgcuguaagccaugc |
| REST- | | gcauggcuuacagcg | | caatatgaagcagaa | | caauaugaagcagaa |
| gRN | | | | | | |
| A5 | | | | | | |
| human | 88 | cuuagcacugugaac | 124 | gtgcatcacatcaga | 160 | gugcaucacaucaga |
| REST- | | ucugaugugaugcac | | gttcacagtgctaag | | guucacagugcuaag |
| gRN | | | | | | |
| A6 | | | | | | |
| human | 89 | uugccugcuucucug | 125 | gtggaagagagtgca | 161 | guggaagagagugca |
| REST- | | cacucucuuccac | | gagaagcaggcaa | | gagaagcaggcaa |
| gRN | | | | | | |
| A7 | | | | | | |
| human | 90 | cagcggucacagcga | 126 | ctccaagggccccat | 162 | cuccaagggccccau |
| REST- | | auggggcccuuggag | | tcgctgtgaccgctg | | ucgcugugaccgcug |
| gRN | | | | | | |
| A8 | | | | | | |
| human | 91 | cucauuauccccagc | 127 | gaaacaccacaccag | 163 | gaaacaccacaccag |
| REST- | | ucucauuauccccag | | agctggggataatga | | agcuggggauaauga |
| gRN | | cu | | g | | g |
| A9 | | | | | | |
| human | 92 | ugauacucgcucacu | 128 | gcacatacacaacag | 164 | gcacauacacaacag |
| REST- | | guuguguaugugc | | tgagcgagtatca | | ugagcgaguauca |
| gRN | | | | | | |
| A10 | | | | | | |
| human | 93 | cguucuccuguauga | 129 | cagcatgttagaact | 165 | cagcauguuagaacu |
| REST- | | guucuaacaugcug | | catacaggagaacg | | cauacaggagaacg |
| gRN | | | | | | |
| A11 | | | | | | |
| human | 94 | augagucuucugaga | 130 | gtccttactcaagtt | 166 | guccuuacucaaguu |
| REST- | | acuugaguaaggac | | ctcagaagactcat | | cucagaagacucau |
| gRN | | | | | | |
| A12 | | | | | | |
| human | 95 | ucuaguuagaugagu | 131 | actcaagttctcaga | 167 | acucaaguucucaga |
| REST- | | cuucugagaacuuga | | agactcatctaacta | | agacucaucuaacua |
| gRN | | gu | | ga | | ga |
| A13 | | | | | | |
| human | 96 | cauaugucuaguuag | 132 | ctcagaagactcatc | 168 | cucagaagacucauc |
| REST- | | augagucuucugag | | taactagacatatg | | uaacuagacauaug |
| gRN | | | | | | |
| A14 | | | | | | |
| human | 97 | aaugaguacgcauau | 133 | catctaactagacat | 169 | caucuaacuagacau |
| REST- | | gucuaguuagaug | | atgcgtactcatt | | augcguacucauu |
| gRN | | | | | | |
| A15 | | | | | | |
| human | 98 | cuucucaccugaaug | 134 | agacatatgcgtact | 170 | agacauaugcguacu |
| REST- | | acuucucaccugaau | | cattcaggtgagaag | | cauucaggugagaag |
| gRN | | ga | | | | |
| A16 | | | | | | |
| human | 99 | cacugaucacauuua | 135 | caggtgagaagccat | 171 | caggugagaagccau |
| REST- | | aauggcuucucaccu | | ttaaatgtgatcagt | | uuaaaugugaucagu |
| gRN | | g | | g | | g |
| A17 | | | | | | |
| human | 100 | aggccacauaacugc | 136 | aaatgtgatcagtgc | 172 | aaaugugaucagugc |
| REST- | | acugaucacauuu | | agttatgtggcct | | aguuauguggccu |
| gRN | | | | | | |
| A18 | | | | | | |
| human | 101 | guuacuucauguuga | 137 | atgtggcctctaatc | 173 | auguggccucuaauc |
| REST- | | uuagaggccacau | | aacatgaagtaac | | aacaugaaguaaC |
| gRN | | | | | | |
| A19 | | | | | | |
| human | 102 | cccauugugaaccug | 138 | aacccgccatgcaag | 174 | aacccgccaugcaag |
| REST- | | ucuugcauggggguu | | acaggttcacaatgg | | acagguucacaaugg |
| gRN | | | | g | | g |
| A20 | | | | | | |
| human | 103 | ugggcaauuaagagg | 139 | cacaatgggcctaaa | 175 | cacaaugggccuaaa |
| REST- | | uuuaggcccauugug | | cctcttaattgccca | | ccucuuaauugccca |
| gRN | | | | | | |
| A21 | | | | | | |
| human | 104 | uaaucacaguguggg | 140 | aaacctcttaattgc | 176 | aaaccucuuaauugc |
| REST- | | caauuaagagguuu | | ccacactgtgatta | | ccacacugugauua |
| gRN | | | | | | |
| A22 | | | | | | |
| human | 105 | auuaggacaaguagg | 141 | caaatctaagcatcc | 177 | caaaucuaagcaucc |
| REST- | | augcuuagauuug | | tacttgtcctaat | | uacuuguccuaau |
| gRN | | | | | | |
| A23 | | | | | | |
| human | 106 | aagauuccucaucau | 142 | acatggtcctgtgaa | 178 | acaugguccugugaa |
| REST- | | ucacaggaccaugu | | tgatgaggaatctt | | ugaugaggaaucuu |
| gRN | | | | | | |
| A24 | | | | | | |
| human | 107 | agcaggccccuccug | 143 | cagatggaggtggtt | 179 | cagauggaggugguu |
| REST- | | aaccaccuccaucug | | caggaggggcctgct | | caggaggggccugcu |
| gRN | | | | | | |
| A25 | | | | | | |
| human | 108 | cagcagcuccuucug | 144 | caggaggggcctgct | 180 | caggaggggccugcu |
| REST- | | agcaggccccuccug | | cagaaggagctgctg | | cagaaggagcugcug |
| gRN | | | | | | |
| A26 | | | | | | |
| human | 109 | auuugggcaaccucc | 145 | ggagactgctcagac | 181 | ggagacugcucagac |
| REST- | | gucugagcagucucc | | ggaggttgcccaaat | | ggagguugcccaaau |
| gRN | | | | | | |
| A27 | | | | | | |
| human | 110 | ccucuaugggaggag | 146 | aagatagagctgtct | 182 | aagauagagcugucu |
| REST- | | acagcucuaucuu | | cctcccatagagg | | ccucccauagagg |
| gRN | | | | | | |
| A28 | | | | | | |
| human | 111 | cuaucuuuaacaggc | 147 | gaagttggcttagtg | 183 | gaaguuggcuuagug |
| REST- | | acuaagccaacuuc | | cctgttaaagatag | | ccuguuaaagauag |
| gRN | | | | | | |
| A29 | | | | | | |
| human | 112 | cucucaucugccucu | 148 | cagaaagtaggagca | 184 | cagaaaguaggagca |
| REST- | | ucugcuccuacuuuc | | gaagaggcagatgag | | gaagaggcagaugag |
| gRN | | ug | | ag | | ag |
| A30 | | | | | | |
| human | 113 | ccauuuaaaguuuca | 149 | gaatacgccagaggg | 185 | gaauacgccagaggg |
| REST- | | ccccucuggcguauu | | tgaaactttaaatgg | | ugaaacuuuaaaugg |
| gRN | | c | | | | |
| A31 | | | | | | |
| human | 114 | cuauacugucagucu | 150 | aaatggtaaacatca | 186 | aaaugguaaacauca |
| REST- | | gauguuuaccauuu | | gactgacagtatag | | gacugacaguauag |
| gRN | | | | | | |
| A32 | | | | | | |
| human | 115 | uucuucuacugcuga | 151 | caccaatgcttcccc | 187 | caccaaugcuucccc |
| REST- | | agggggaagcauugg | | cttcagcagtagaag | | cuucagcaguagaag |
| gRN | | ug | | aa | | aa |
| A33 | | | | | | |
| human | 116 | caggaggugaugcca | 152 | gtccaaaactgcact | 188 | guccaaaacugcacu |
| REST- | | gugcaguuuuggac | | ggcatcacctcctg | | ggcaucaccuccug |
| gRN | | | | | | |
| A34 | | | | | | |
| human | 117 | uggaacuggccgagc | 153 | ctggattgcatgggg | 189 | cuggauugcaugggg |
| REST- | | cccaugcaauccag | | ctcggccagttcca | | cucggccaguucca |
| gRN | | | | | | |
| A35 | | | | | | |
| human | 118 | cugaaagaacgauca | 154 | gtttgtatcttctgt | 190 | |
| REST- | | cagaagauacaaac | | gatcgttctttcag | | |
| gRNA36 | | | | | | |

### Mouse REST Amino Acid Sequence (SEQ ID NO: 191):

MATQVMGQSSGGGSLFNNSANMGMALTNDMYDLHELSKAELAAPQ
LIMLANVALTGEASGSCCDYLVGEERQMAELMPVGDNHFSESEGE
GLEESADLKGLENMELGSLELSAVEPQPVFEASAAPEIYSANKDP
APETPVAEDKCRSSKAKPFRCKPCQYEAESEEQFVHHIRIHSAKK
FFVEESAEKQAKAWESGSSPAEEGEFSKGPIRCDRCGYNTNRYDH
YMAHLKHHLRAGENERIYKCIICTYTTVSEYHWRKHLRNHFPRKV
YTCSKCNYFSDRKNNYVQHVRTHTGERPYKCELCPYSSSQKTHLT
RHMRTHSGEKPFKCDQCNYVASNQHEVTRHARQVHNGPKPLNCPH
CDYKTADRSNFKKHVELHVNPRQFNCPVCDYAASKKCNLQYHFKS
KHPTCPSKTMDVSKVKLKKTKKREADLLNNAVSNEKMENEQTKTK
GDVSGKKNEKPVKAVGKDASKEKKPGSSVSVVQVTTRTRKSAVAA
ETKAAEVKHTDGQTGNNPEKPCKAKKNKRKKDAEAHPSEEPVNEG
PVTKKKKKSECKSKIGTNVPKGGGRAEERPGVKKQSASLKKGTKK
TPPKTKTSKKGGKLAPKGMGQTEPSSGALAQVGVSPDPALIQAEV
TGSGSSQTELPSPMDIAKSEPAQMEVSLTGPPPVEPAQMEPSPAK
PPQVEAPTYPQPPQRGPAPPTGPAPPTGPAPPTEPAPPTGLAEME
PSPTEPSQKEPPPSMEPPCPEELPQAEPPPMEDCQKELPSPVEPA
QIEVAQTAPTQVQEEPPPVSEPPRVKPTKRSSLRKDRAEKELSLL
SEMARQEQVLMGVGLVPVRDSKLLKGNKSAQDPPAPPSPSPKGNS
REETPKDQEMVSDGEGTIVFPLKKGGPEEAGESPAELAALKESAR
VSSSEQNSAMPEGGASHSKCQTGSSGLCDVDTEQKTDTVPMKDSA
AEPVSPPTPTVDRDAGSPAVVASPPITLAENESQEIDEDEGIHSH
DGSDLSDNMSEGSDDSGLHGARPTPPEATSKNGKAGLAGKVTEGE
FVCIFCDRSFRKEKDYSKHLNRHLVNVYFLEEAAEEQEEQEEREE
QE*

### Mouse REST Coding Sequence (SEQ ID NO: 192):

atggccacccaggtgatggggcagtcttctggaggaggcagtctc
ttcaacaacagtgccaacatgggcatggccttaaccaacgacatg
tacgacctgcacgagctctcgaaagctgaactggcagcccctcag
ctcatcatgttagccaacgtggccctgacgggggaggcaagcggc
agctgctgcgattacctggtcggtgaagagaggcagatggccgaa
ttgatgcccgtgggagacaaccacttctcagaaagtgaaggagaa
ggcctggaagagtcggctgacctcaaagggctggaaaacatggaa
ctgggaagtttggagctaagtgctgtagaaccccagcccgtattt
gaagcctcagctgccccagaaatatacagcgccaataaagatccc
gctccagaaacacccgtggcggaagacaaatgcaggagttctaag
gccaagcccttccggtgtaagccttgccagtacgaagccgaatct
gaagagcagtttgtgcatcacatccggattcacagcgctaagaag
ttctttgtggaggaaagtgcagagaaacaggccaaagcctgggag
tcggggtcgtctccggccgaagagggcgagttctccaaaggcccc
atccgctgtgaccgctgtggctacaataccaaccggtatgaccac
tacatggcacacctgaagcaccacctgcgagctggcgagaacgag
cgcatctacaagtgcatcatctgcacgtacacgacggtcagcgag
taccactggaggaaacacctgagaaaccatttccccaggaaagtc
tacacctgcagcaagtgcaactacttctcagacagaaaaaataac
tacgttcagcacgtgcgaactcacacaggagaacgcccgtataaa
tgtgaactttgtccttactcaagctctcagaagactcatctaacg
cgacacatgcggactcattcaggtgagaagccatttaaatgtgat
cagtgcaattatgtggcctctaatcagcatgaagtgacccgacat
gcaagacaggttcacaacgggcctaaacctcttaattgcccgcac
tgtgactacaaaacagcagatagaagcaacttcaaaaagcacgtg
gagctgcatgttaacccacggcagttcaactgccccgtgtgtgac
tacgcggcttctaagaagtgtaatctacaataccatttcaaatct
aagcatcccacctgtcccagcaaaacaatggatgtctccaaagtg
aagctaaagaaaaccaaaaagagagaggctgacctgcttaataac
gccgtcagcaacgagaagatggagaatgagcaaacaaaaacaaag
ggggatgtgtctgggaagaagaacgagaaacctgtaaaagctgtg
ggaaaagatgcttcaaaagagaagaagcctggtagcagtgtctca
gtggtccaggtaactaccaggactcggaagtcagcggtggcggcg
gagactaaagcagcagaggtgaaacacacagacggacaaacagga
aacaatccagaaaagccctgtaaagccaagaaaaacaaaagaaag
aaggatgctgaggcccatccctccgaagagcctgtgaacgaggga
ccagtgacaaaaaagaaaaagaagtctgagtgcaaatcaaaaatc
ggtaccaacgtgccaaagggcggcggccgagcggaggagaggccg
ggggtcaagaagcaaagcgcttcccttaagaaaggcacaaagaag
acgccgcccaagacaaagacaagtaaaaaaggtggcaaacttgct
ccaaaggggatggggcagacagaaccttcttctggggcattggct
caagtgggggtgtctccagaccctgccctcattcaggcagaggtc
accgggtcaggatcttctcagacagagcttccttcacccatggat
attgctaagtcagagcccgcccagatggaggtttccctaacaggg
ccacctccggtggagcctgctcaaatggagccatcgcctgcgaaa
cctccccaggtagaagcacccacttacccccagcctccccaaagg
gggcctgcccctcccacggggcctgcccctcccacggggcctgcc
cctcccacggagcctgcccctcccacggggcttgccgagatggaa
ccttctcccacggagccttcccagaaggaaccacctcccagtatg
gagcctccctgccccgaggagctgcctcaggccgagccacctcct
atggaggattgtcagaaggagctgccttctcccgtggagcccgct
cagattgaggttgctcagacggcccctacgcaggttcaggaggag
ccccctcctgtctcggagccacctcgggtgaagccaaccaaaaga
tcatctctccggaaagacagagcagagaaggagctgagcctgctg
agtgagatggcgcggcaggagcaggtcctcatgggggttggcttg
gtgcctgttagagacagcaagcttctgaagggaaacaagagcgcc
caggaccccccagccccaccgtcaccatcgccaaagggaaactcg
agggaagagacacccaaggaccaagaaatggtctctgatggggaa
ggaactatagtattccctctcaagaaaggaggaccagaggaagct
ggagagagtccagctgagttggctgctctcaaggagtctgcccgt
gtttcatcctctgaacaaaactcagccatgccagagggtggagca
tcacacagcaagtgtcagactggctcctctgggctttgtgacgtg
gacactgagcagaagacagatactgtccccatgaaagactccgca
gcagagccagtgtcccctcctaccccaacagtggaccgtgacgca
gggtcaccagctgtagtggcctcccctcctatcacgttggctgaa
aacgagtctcaggaaattgatgaagatgaaggcatccatagccat
gatggaagtgacctgagtgacaacatgtctgaggggagtgacgac
tcaggactgcacggggctcggccgacaccaccagaagctacgtca
aaaaatgggaaggcagggttggctggtaaagtgactgagggagag
tttgtgtgtattttctgtgatcgttcttttagaaaggaaaaagat
tatagcaaacacctcaatcgccacttggtgaatgtgtacttccta
gaagaagcagctgaggagcaggaggagcaggaggagcgggaggag
caggagtag

### EXAMPLE

### Example 1: MiR124 Incapable of Transdifferentiating Glial Cells into Neurons or Dopamine Neurons

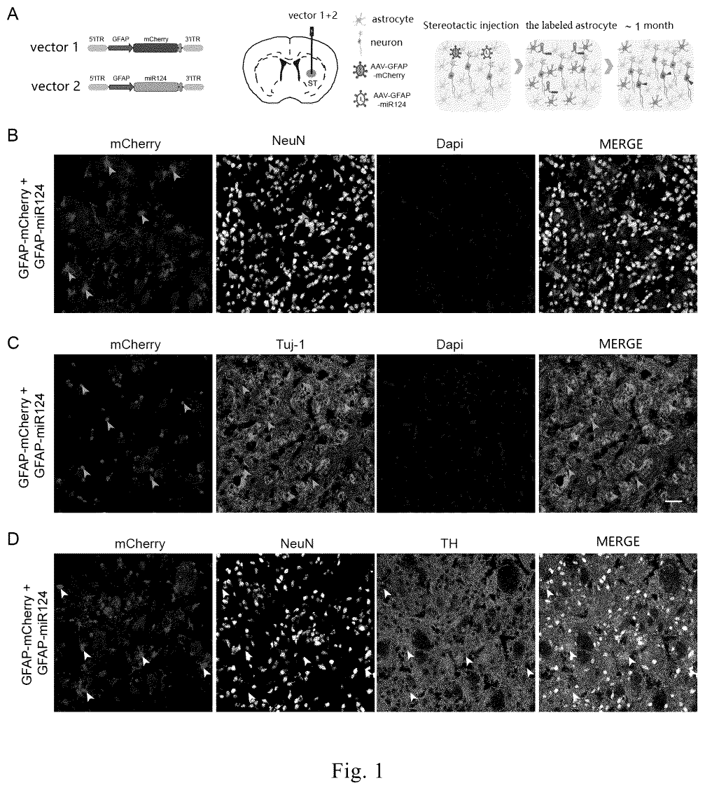

Previous studies have showed that overexpression of miR124 could differentiate stem cells into neurons, and further studies showed that Ptbp1 could transdifferentiate glial cells into neurons via miR124-mediated transdifferentiation. However, these are in-vitro studies. In order to investigate whether miR124 can transdifferentiate glial cells into neurons in animals, an AAV vector that could specifically express miR124 in glial cells was constructed in this study, and it was investigated whether miR124 could transdifferentiate glial cells into neurons in vivo by injecting the AAV overexpressing miR124 in mouse brain (FIG. 1A). Approximately 1 month after injection, materials were obtained and analyzed. It could be found that, unlike the in-vitro studies, fluorescent protein-labeled cells in red were not co-labeled with the neuron-specific marker NeuN (FIG. 1B). This indicated that overexpression of miR124 could not transdifferentiate glial cells into neurons in mice. To further investigate whether cells overexpressing miR124 are in the neonatal neuronal stage, the cells were stained with a neonatal neuron-specific protein marker Tuj-1. It was found that the fluorescently labeled cells in red were co-labeled with Tuj-1 (FIG. 1C). This indicated that overexpression of miR 124 could not directly transdifferentiate astrocytes into neurons. It was found in staining analysis with a dopamine-specific protein marker TH that red cells over-expressing miR124 also did not express TH. This indicated that overexpression of miR124 could not transdifferentiate glial cells into dopamine neurons (FIG. 1D). The above results show that overexpression of miR 124 in vivo cannot transdifferentiate glial cells into neurons or dopamine neurons.

### Example 2: Picking Out gRNAs Efficiently Targeting REST in N2A Cells

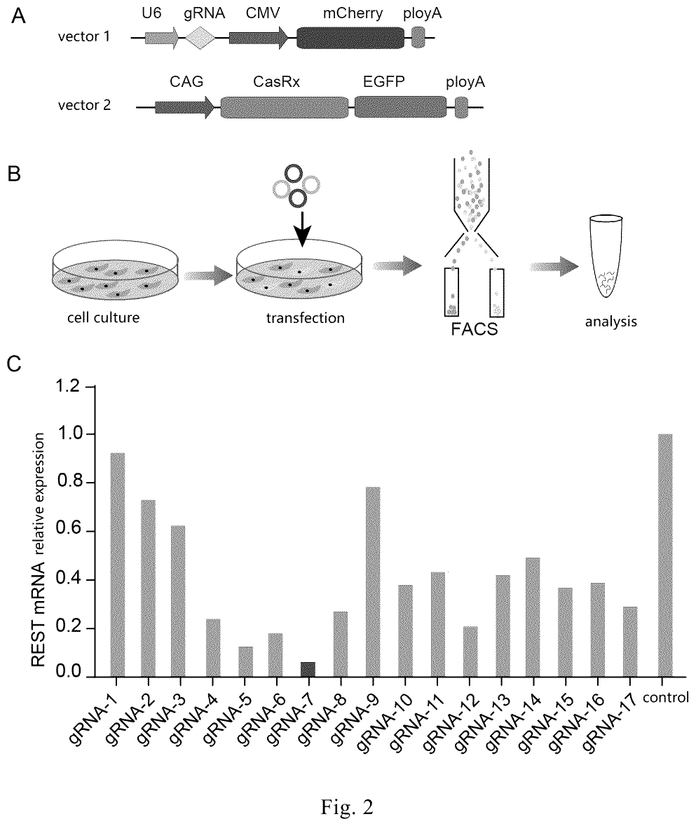

In order to pick out gRNAs for CasRx to efficiently target REST, firstly, 17 gRNAs targeting REST (see SEQ ID NOs: 4-54) were designed and constructed onto a U6-gRNA-CMV-mCherry vector, and different gRNAs were co-transformed into N2A cells with a CAG-CasRx-P2A-EGFP plasmid, respectively (FIGS. 2A and 2B). 48 hours after cell transfection, the transfected GFP and mCherry double-positive cells were separated by using fluorescence-activated cell sorting, and the expression level of REST mRNA was measured by Q-PCR, thereby picking out the gRNA with the highest efficiency for targeting REST. QPCR results indicated that most gRNAs could efficiently knock down the level of REST mRNA, with gRNA-7 being the most efficient and capable of knocking down approximately 94% expression level of REST mRNA (FIG. 2C).

### Example 3: Transdifferentiation of Astrocytes into Neurons In Vivo

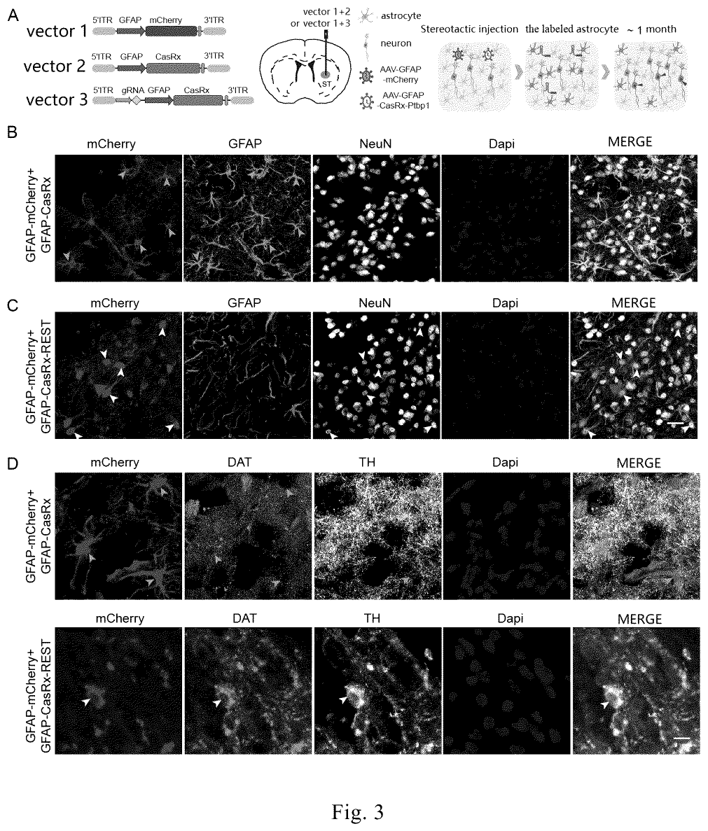

Previous studies have shown that in-vitro knockdown of REST expression could transdifferentiate fibroblasts into neurons, although the transdifferentiation efficiency was only about 5%. However, in a complex environment in vivo, whether knockdown of REST expression could achieve a transdifferentiation process or not is a question. To further investigate whether CasRx-mediated REST knockdown technique could achieve the transdifferentiation of glial cells into neurons in vivo, AAV expression vectors were constructed and packaged, and then the AAV vectors were injected into the striatum of mouse brain (FIG. 3A). To label glial cells, mCherry expression was driven by a glial cell-specific promoter GFAP. To specifically express CasRx in glial cells, CasRx expression was also driven by the glial cell-specific promoter GFAP. The virus injected in the control group was a mixed AAV of GFAP-mCherry and GFAP-CasRx, wherein mCherry could label infected glial cells; the AAV combination injected in the experimental group was GFAP-mCherry+GFAP-CasRx-REST (expressing gRNA-7), wherein GFAP-CasRx-REST could specifically target REST mRNA (FIG. 3A). Analysis was performed approximately 1 month after AAV injection. It was found that the fluorescently labeled cells in red of the control group remained co-labeled with the glial cell-specific protein marker GFAP, but not with those stained with the neuron-specific marker NeuN (FIG. 3B). However, in the group injected with GFAP-mCherry+GFAP-CasRx-REST, a large number of mCherry positive cells were found to be co-labeled with NeuN, but not with those stained with GFAP (FIG. 3C). These results indicated that targeted knockdown of REST expression could efficiently transdifferentiate astrocytes into neurons in mice. To further investigate whether knocking down REST could transdifferentiate glial cells into dopamine neurons, the dopamine neuron-specific cell markers TH and DAT were adopted in this study for staining. In the control group injected with GFAP-mCherry+GFAP-CasRx, fluorescently labeled cells in red expressed neither TH nor DAT, while in the group injected with AAVs capable of knocking down REST (GFAP-mCherry+GFAP-CasRx-REST), a small fraction of fluorescently labeled cells in red expressed the dopamine neuron-specific cell markers TH and DAT (FIGS. 3D and 3E). The above results indicated that knockdown of REST expression in the striatum could transdifferentiate astrocytes into dopamine neurons.

### Example 4: Inhibition Against REST Gene Expression Using Epigenetic Method

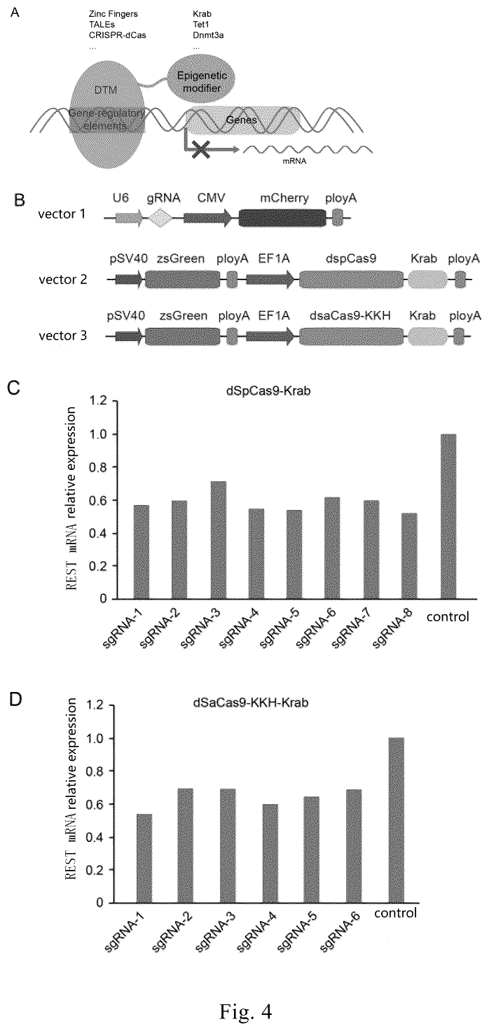

Epigenetic modification is also a common method for manipulating gene expression, and in order to investigate whether the epigenetic method can effectively inhibit the expression of REST mRNA, DNA binding proteins (e.g., Zinc fingers, TALEs, CRISPR-dCas, etc.) and epigenetic regulatory elements (e.g., KRAB, Dnmt3a, Tet1, etc.) were expressed by fusion via flexible linker amino acids (FIG. 4A). The DNA targeting proteins used in this study were two different CRISPR-dCas (dSpCas9, dSaCas9-KKH), were subjected to fusion expression together with an epigenetic modification protein Krab inhibitory domain, were used for fluorescence-activated cell sorting by driving expression of EGFP proteins by SV40, and were used for fluorescence-activated cell sorting by driving mCherry fluorescence expression by CMV in the same plasmid vector of U6-gRNA, the gRNA being independently driven by U6 (FIG. 4B). 48 hours after N2A cell transfection, it was found that both dSpCas9-KRAB and dSaCas9-KKH-Krab could effectively reduce the expression of REST mRNA by Q-PCR, and most REST-targeting sgRNAs (see SEQ ID NOs: 55-82) could reduce the level of REST mRNA to about half of the original level (FIGS. 4C and 4D).

### Example 5: Efficient CasRx-Mediated REST Knockdown in Human Cells

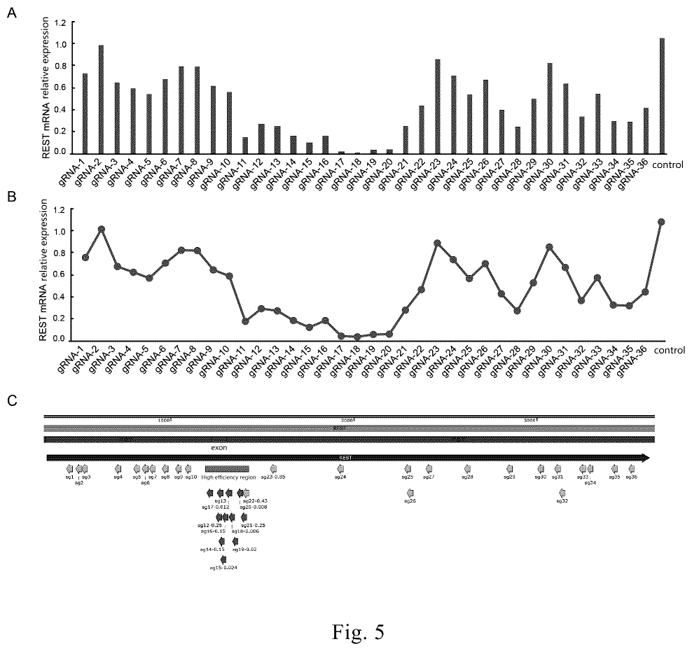

To further investigate whether REST expression could be efficiently knocked down in human cells, a series of gRNAs targeting human REST mRNA were constructed (see SEQ ID NOs: 83-190). Different gRNA knockdown efficiencies were measured by Q-PCR, and it was found that most gRNAs could effectively knock down REST mRNA (FIG. 5A). Analysis of these gRNA positions found that the gRNAs with a very high knockdown efficiency were concentrated in a small region of REST mRNA (FIGS. 5B and 5C). The results showed that the region is a preferred position for designing gRNA targeting.

### Example 6: gRNAs Efficiently Targeting Human Capable of Achieving Efficient REST Knockdown in Non-Human Primates and Mice

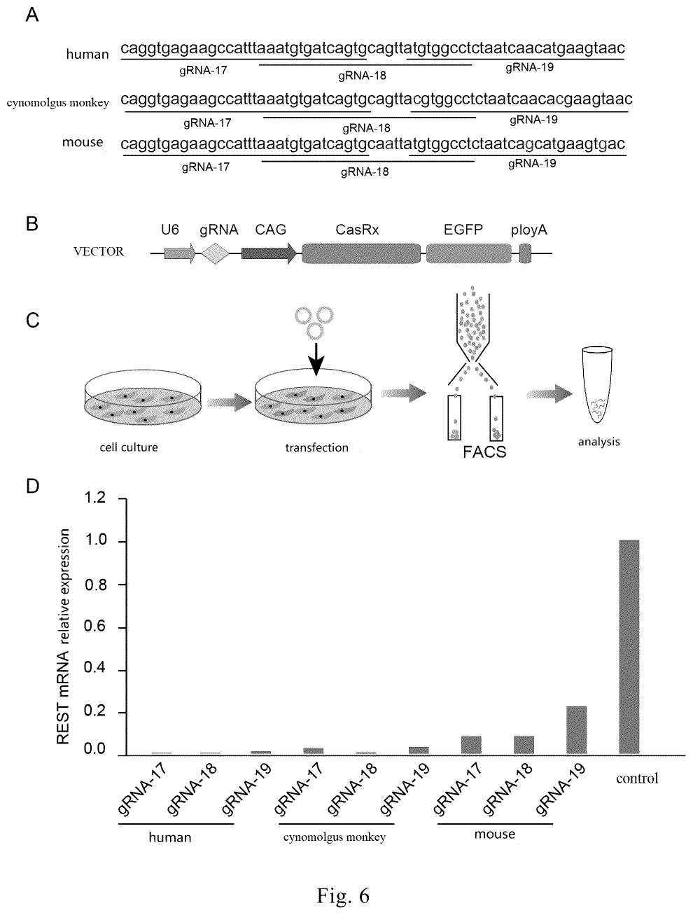

To investigate whether gRNAs efficiently targeting humans could also efficiently target non-human primates or mice, 3 gRNAs were selected in this study from the gRNAs efficiently targeting human REST genes that had been picked out for testing (gRNA 17, gRNA 18, and gRNA 19). The gRNA-17 sequences were homologous in humans, non-human primates and mice, and the sequences were completely consistent: gRNA-18 had 1 base mismatch in cynomolgus monkeys and mice, and gRNA-19 had 2 base mismatches in cynomolgus monkeys and mice (FIG. 6A). As shown in FIG. 6B, in this study, gRNAs and CasRx were constructed into the same expression plasmid, and after 293T, Cos-7 and N2A cells were transfected with the plasmid, the transfected positive cells were separated by using fluorescence-activated cell sorting, and the difference in the expression level of REST mRNA was detected by QPCR (FIG. 6C). The results showed that all of the 3 human-targeting gRNAs could efficiently target REST in non-human primates and mice, and could also effectively knock down the expression level of REST mRNA in non-human primates and mice (FIG. 6D). The above results show that the gRNAs of the present invention can be applied to different species while achieving the technical effects of the present invention as well.

### Example 7: CasRx-gRNA System Targeting Human REST Capable of Transdifferentiating Glial Cells into Neurons in Mice

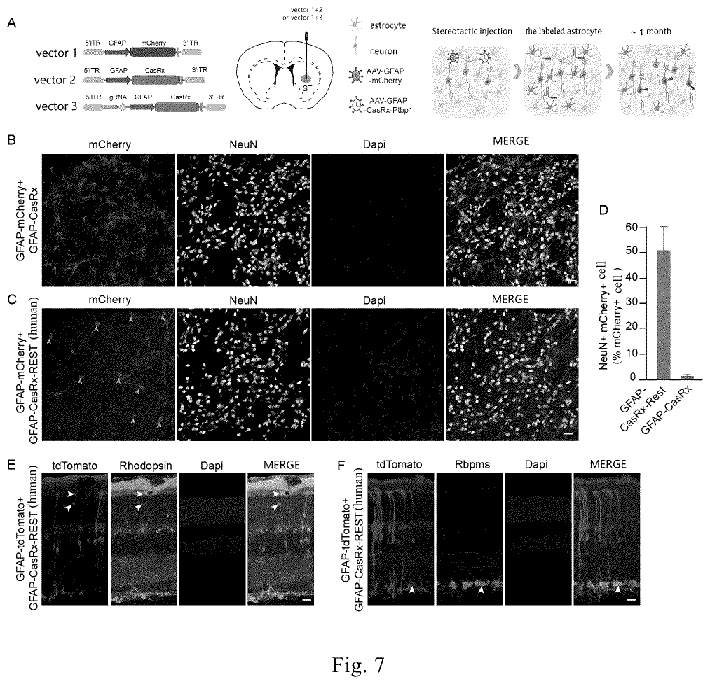

To investigate whether human-targeting gRNAs could efficiently transdifferentiate glial cells into neurons, in this study, human-targeting gRNA-17 (gRNA (human)) and CasRx were constructed into AAV vectors and the AAV vectors were packaged. GFAP-CasRx-REST and GFAP-mCherry were co-injected into the mouse brain, and GFAP-CasRx+GFAP-mCherry was injected into the mice in the control group, and then analysis was performed 1 month after injection (FIG. 7A). The results showed that gRNAs targeting human REST could transdifferentiate astrocytes into neurons, fluorescently labeled cells in red were co-labeled with the neuron-specific protein marker NeuN (50.71%±11.12%, SEM, 3 mice per group), while fluorescently labeled cells in red in the mouse brain injected with the AAV of the control group still exhibited typical glial cell morphology and were not co-labeled with NeuN (FIGS. 7B, 7C and 7D). The results show that the CasRx-gRNA system targeting human REST can efficiently transdifferentiate glial cells into neurons, and has the potential of treating diseases associated with the loss of neurons. To further investigate whether knocking down REST in the retina could transdifferentiate Müller glia into functional neurons in the retina, such as photoreceptor cells or retinal ganglion cells, C57 mice at about 5 weeks of age were injected subretinally with an AAV vector of GFAP-tdTomato and GFAP-CasRx-REST (previous documents showed that GFAP could be used as a Müller glia-specific promoter, and GFAP-tdTomato was used to label Müller glia), and after knockdown of REST expression in Müller glia in the retina, cells in the outer granular layer of the retina were found to express both Rhodopsin and tdTomato, while cells in the retinal ganglion cell layer were found to express both Rbpms and tdTomato. These data suggested that knocking down REST in the retina could transdifferentiate Müller glia into photoreceptor cells or retinal ganglion cells, respectively (FIGS. 7E and 7F).

### REFERENCES

1. Mccarthy, K. D. & Devellis, J. Preparation Of Separate Astroglial And Oligodendroglial Cell-Cultures From Rat Cerebral Tissue. Journal Of Cell Biology 85, 890-902 (1980).
2. Zhou, H. et al. Cerebellar modules operate at different frequencies. Elife 3, e02536 (2014).
3. Xu, H. T. et al. Distinct lineage-dependent structural and functional organization of the hippocampus. Cell 157, 1552-1564 (2014).
4. Su, J. et al. Reduction of HIP2 expression causes motor function impairment and increased vulnerability to dopaminergic degeneration in Parkinson's disease models. Cell Death Dis 9, 1020 (2018).
5. Chavez, A. et al. Comparison of Cas9 activators in multiple species. Nat Methods 13, 563-567 (2016).
6. Qian, Hao et al. Reversing a model of Parkinson's disease with in situ converted nigral neurons. Nature 582, 550-556 (2020).
7. Zhou, Haibo et al. Glia-to-Neuron Conversion by CRISPR-CasRx Alleviates Symptoms of Neurological Disease in Mice. Cell 181,590-603.e16 (2020).

All documents mentioned in the present disclosure are incorporated by reference in the present application as if each was individually incorporated by reference. Furthermore, it should be understood that various changes or modifications of the present disclosure can be made by those skilled in the art after reading the above teachings of the present disclosure, and these equivalents also fall within the scope of the appended claims of the present application.

1.-39. (canceled)

40. A method for producing functional dopamine neurons from glial cells, comprising transdifferentiating or reprogramming the glial cells into functional dopamine neurons by using a REST inhibitor, wherein the REST inhibitor reduces the expression or activity of a REST gene, an RNA thereof, or an encoding protein thereof, wherein, the glial cells are astrocytes from striatum.

41. The method of claim 40, wherein the REST inhibitor could be used to prevent and/or treat a disease associated with loss of function or death of functional dopamine neurons.

42. The method of claim 41, wherein the disease associated with loss of function or death of functional dopamine neurons is a nervous system disease selected from the group consisting of stroke, Parkinson's disease, schizophrenia, and depression.

43. A method for producing functional retinal ganglion cells (RGCs) or photoreceptor cells from Müller glia (MG), comprising transdifferentiating or reprogramming Müller glia into functional RGCs or photoreceptor cells by using a REST inhibitor, wherein the REST inhibitor reduces the expression or activity of a REST gene, an RNA thereof, or an encoding protein thereof;
wherein the Müller glia are from retina, and wherein the photoreceptor cells comprise rod cells and cone cells.

44. A method of claim 43, wherein the REST inhibitor could prevent or treat a visual system disease associated with loss of function or death of RGCs or photoreceptor cells.
preferably, the REST inhibitor is formulated for administration to a visual system, preferably a subretinal space or a vitreous cavity.

45. The method of claim 44, wherein the visual system disease associated with loss of function or death of RGCs is selected from the group consisting of visual impairment due to death of RGCs, glaucoma, age-related RGC pathology, optic nerve damage, retinal ischemia or hemorrhage, Leber hereditary optic neuropathy, and a combination thereof; and wherein the visual system disease associated with loss of function or death of photoreceptor cells is selected from: photoreceptor cell degeneration or death due to damage or degenerative diseases, macular degeneration, retinitis pigmentosa, diabetes-related blindness, night blindness, color blindness, inherited blindness, congenital amaurosis, and a combination thereof.

46. The method of claim 40, wherein the REST inhibitor is selected from: antibodies, small molecule compounds, microRNA, siRNA, shRNA, antisense oligonucleotides, REST binding proteins and protein domains, polypeptides, aptamers, gene editors, PROTACs, epigenetic regulators, and a combination thereof.

47. The method of claim 46, wherein the REST inhibitor comprises:
(a) a gene-editing protein or an expression vector thereof, and an editing system selected from the group consisting of a CRISPR system, a ZFN system, a TALEN system, an RNA-editing system, and a combination thereof, and (b) one or more gRNAs or an expression vector thereof, wherein the gRNA is a DNA or an RNA guiding the gene-editing protein to specifically bind to a REST gene.

48. The method of claim 47, wherein the gRNA guides the gene-editing protein to specifically bind to nucleotides at positions 867-1103 (SEQ ID NO: 3) of REST coding sequence.

49. The method of claim 47, wherein the gRNA comprises a sequence selected from SEQ ID NOs: 4-20 and 83-118 or comprises a sequence encoded by sequences set forth in SEQ ID NOs: 55-62 and 71-76.


SEQUENCE LISTING (202 sequences)

SEQ ID NO: 1 - protein, 1097 aa, Homo sapiens
MATQVMGQSSGGGGLFTSSGNIGMALPNDMYDLHDLSKAELAAPQLIMLANVALTGEVNGSCCDYLVGEERQMAELMPVGDNNFSDSEEGEGLEESADIKGEPHGLENMELRSLELSVVEPQPVFEASGAPDIYSSNKDLPPETPGAEDKGKSSKTKPFRCKPCQYEAESEEQFVHHIRVHSAKKFFVEESAEKQAKARESGSSTAEEGDFSKGPIRCDRCGYNTNRYDHYTAHLKHHTRAGDNERVYKCIICTYTTVSEYHWRKHLRNHFPRKVYTCGKCNYFSDRKNNYVQHVRTHTGERPYKCELCPYSSSQKTHLTRHMRTHSGEKPFKCDQCSYVASNQHEVTRHARQVHNGPKPLNCPHCDYKTADRSNFKKHVELHVNPRQFNCPVCDYAASKKCNLQYHFKSKHPTCPNKTMDVSKVKLKKTKKREADLPDNITNEKTEIEQTKIKGDVAGKKNEKSVKAEKRDVSKEKKPSNNVSVIQVTTRTRKSVTEVKEMDVHTGSNSEKFSKTKKSKRKLEVDSHSLHGPVNDEESSTKKKKKVESKSKNNSQEVPKGDSKVEENKKQNTCMKKSTKKKTLKNKSSKKSSKPPQKEPVEKGSAQMDPPQMGPAPTEAVQKGPVQVEPPPPMEHAQMEGAQIRPAPDEPVQMEVVQEGPAQKELLPPVEPAQMVGAQIVLAHMELPPPMETAQTEVAQMGPAPMEPAQMEVAQVESAPMQVVQKEPVQMELSPPMEVVQKEPVQIELSPPMEVVQKEPVKIELSPPIEVVQKEPVQMELSPPMGVVQKEPAQREPPPPREPPLHMEPISKKPPLRKDKKEKSNMQSERARKEQVLIEVGLVPVKDSWLLKESVSTEDLSPPSPPLPKENLREEASGDQKLLNTGEGNKEAPLQKVGAEEADESLPGLAANINESTHISSSGQNLNTPEGETLNGKHQTDSIVCEMKMDTDQNTRENLTGINSTVEEPVSPMLPPSAVEEREAVSKTALASPPATMAANESQEIDEDEGIHSHEGSDLSDNMSEGSDDSGLHGARPVPQESSRKNAKEALAVKAAKGDFVCIFCDRSFRKGKDYSKHLNRHLVNVYYLEEAAQGQE

SEQ ID NO: 2 - DNA, 3294 bp, Homo sapiens
atggccacccaggtaatggggcagtcttctggaggaggagggctgtttaccagcagtggcaacattggaatggccctgcctaacgacatgtatgacttgcatgacctttccaaagctgaactggccgcacctcagcttattatgctggcaaatgtggccttaactggggaagtaaatggcagctgctgtgattacctggtcggtgaagaaagacagatggcagaactgatgccggttggggataacaacttttcagatagtgaagaaggagaaggacttgaagagtctgctgatataaaaggtgaacctcatggactggaaaacatggaactgagaagtttggaactcagcgtcgtagaacctcagcctgtatttgaggcatcaggtgctccagatatttacagttcaaataaagatcttccccctgaaacacctggagcggaggacaaaggcaagagctcgaagaccaaaccctttcgctgtaagccatgccaatatgaagcagaatctgaagaacagtttgtgcatcacatcagagttcacagtgctaagaaattttttgtggaagagagtgcagagaagcaggcaaaagccagggaatctggctcttccactgcagaagagggagatttctccaagggccccattcgctgtgaccgctgcggctacaatactaatcgatatgatcactatacagcacacctgaaacaccacaccagagctggggataatgagcgagtctacaagtgtatcatttgcacatacacaacagtgagcgagtatcactggaggaaacatttaagaaaccattttccaaggaaagtatacacatgtggaaaatgcaactatttttcagacagaaaaaacaattatgttcagcatgttagaactcatacaggagaacgcccatataaatgtgaactttgtccttactcaagttctcagaagactcatctaactagacatatgcgtactcattcaggtgagaagccatttaaatgtgatcagtgcagttatgtggcctctaatcaacatgaagtaacccgccatgcaagacaggttcacaatgggcctaaacctcttaattgcccacactgtgattacaaaacagcagatagaagcaacttcaaaaaacatgtagagctacatgtgaacccacggcagttcaattgccctgtatgtgactatgcagcttccaagaagtgtaatctacagtatcacttcaaatctaagcatcctacttgtcctaataaaacaatggatgtctcaaaagtgaaactaaagaaaaccaaaaaacgagaggctgacttgcctgataatattaccaatgaaaaaacagaaatagaacaaacaaaaataaaaggggatgtggctggaaagaaaaatgaaaagtccgtcaaagcagagaaaagagatgtctcaaaagagaaaaagccttctaataatgtgtcagtgatccaggtgactaccagaactcgaaaatcagtaacagaggtgaaagagatggatgtgcatacaggaagcaattcagaaaaattcagtaaaactaagaaaagcaaaaggaagctggaagttgacagccattctttacatggtcctgtgaatgatgaggaatcttcaacaaaaaagaaaaagaaggtagaaagcaaatccaaaaataatagtcaggaagtgccaaagggtgacagcaaagtggaggagaataaaaagcaaaatacttgcatgaaaaaaagtacaaagaagaaaactctgaaaaataaatcaagtaagaaaagcagtaagcctcctcagaaggaacctgttgagaagggatctgctcagatggaccctcctcagatggggcctgctcccacagaggcggttcagaaggggcccgttcaggtggagccgccacctcccatggagcatgctcagatggagggtgcccagatacggcctgctcctgacgagcctgttcagatggaggtggttcaggaggggcctgctcagaaggagctgctgcctcccgtggagcctgctcagatggtgggtgcccaaattgtacttgctcacatggagctgcctcctcccatggagactgctcagacggaggttgcccaaatggggcctgctcccatggaacctgctcagatggaggttgcccaggtagaatctgctcccatgcaggtggtccagaaggagcctgttcagatggagctgtctcctcccatggaggtggtccagaaggagcctgttcagatagagctgtctcctcccatggaggtggtccagaaggaacctgttaagatagagctgtctcctcccatagaggtggtccagaaggagcctgttcagatggagttgtctcctcccatgggggtggttcagaaggagcctgctcagagggagccacctcctcccagagagcctccccttcacatggagccaatttccaaaaagcctcctctccgaaaagataaaaaggaaaagtctaacatgcagagtgaaagggcacggaaggagcaagtccttattgaagttggcttagtgcctgttaaagatagctggcttctaaaggaaagtgtaagcacagaggatctctcaccaccatcaccaccactgccaaaggaaaatttaagagaagaggcatcaggagaccaaaaattactcaacacaggtgaaggaaataaagaagcccctcttcagaaagtaggagcagaagaggcagatgagagcctacctggtcttgctgctaatatcaacgaatctacccatatttcatcctctggacaaaacttgaatacgccagagggtgaaactttaaatggtaaacatcagactgacagtatagtttgtgaaatgaaaatggacactgatcagaacacaagagagaatctcactggtataaattcaacagttgaagaaccagtttcaccaatgcttcccccttcagcagtagaagaacgtgaagcagtgtccaaaactgcactggcatcacctcctgctacaatggcagcaaatgagtctcaggaaattgatgaagatgaaggcatccacagccatgaaggaagtgacctaagtgacaacatgtcagagggtagtgatgattctggattgcatggggctcggccagttccacaagaatctagcagaaaaaatgcaaaggaagccttggcagtcaaagcggctaagggagattttgtttgtatcttctgtgatcgttctttcagaaagggaaaagattacagcaaacacctcaatcgccatttggttaatgtgtactatcttgaagaagcagctcaagggcaggagtaa

SEQ ID NO: 3 - DNA, 237 bp, Homo sapiens
caattatgttcagcatgttagaactcatacaggagaacgcccatataaatgtgaactttgtccttactcaagttctcagaagactcatctaactagacatatgcgtactcattcaggtgagaagccatttaaatgtgatcagtgcagttatgtggcctctaatcaacatgaagtaacccgccatgcaagacaggttcacaatgggcctaaacctcttaattgcccacactgtgatta

SEQ ID NO: 4 - RNA, 30 nt, Artificial Sequence
  gRNA
agcucgugcaggucguacaugucguugguu

SEQ ID NO: 5 - RNA, 31 nt, Artificial Sequence
  gRNA
cgcuguauauuucuggggcagcugaggcuuc

SEQ ID NO: 6 - RNA, 31 nt, Artificial Sequence
  gRNA
cuuuggccuguuucucugcacuuuccuccac

SEQ ID NO: 7 - RNA, 30 nt, Artificial Sequence
  gRNA
cacuugcugcagguguagacuuuccugggg

SEQ ID NO: 8 - RNA, 30 nt, Artificial Sequence
  gRNA
gcguucuccugugugaguucgcacgugcug

SEQ ID NO: 9 - RNA, 30 nt, Artificial Sequence
  gRNA
gaugagucuucugagagcuugaguaaggac

SEQ ID NO: 10 - RNA, 28 nt, Artificial Sequence
  gRNA
gucacuucaugcugauuagaggccacau

SEQ ID NO: 11 - RNA, 30 nt, Artificial Sequence
  gRNA
cgggcaauuaagagguuuaggcccguugug

SEQ ID NO: 12 - RNA, 32 nt, Artificial Sequence
  gRNA
ucacacacggggcaguugaacugccguggguu

SEQ ID NO: 13 - RNA, 25 nt, Artificial Sequence
  gRNA
ugguauuguagauuacacuucuuag

SEQ ID NO: 14 - RNA, 27 nt, Artificial Sequence
  gRNA
cugggacaggugggaugcuuagauuug

SEQ ID NO: 15 - RNA, 25 nt, Artificial Sequence
  gRNA
ucuucucguugcugacggcguuauu

SEQ ID NO: 16 - RNA, 30 nt, Artificial Sequence
  gRNA
gcggcgucuucuuugugccuuucuuaaggg

SEQ ID NO: 17 - RNA, 30 nt, Artificial Sequence
  gRNA
agaagauccugacccggugaccucugccug

SEQ ID NO: 18 - RNA, 30 nt, Artificial Sequence
  gRNA
gcuccauacugggaggugguuccuucuggg

SEQ ID NO: 19 - RNA, 30 nt, Artificial Sequence
  gRNA
aagcuugcugucucuaacaggcaccaagcc

SEQ ID NO: 20 - RNA, 29 nt, Artificial Sequence
  gRNA
aucugucuucugcucaguguccacgucac

SEQ ID NO: 21 - DNA, 30 bp, Mus musculus
aaccaacgacatgtacgacctgcacgagct

SEQ ID NO: 22 - DNA, 31 bp, Mus musculus
gaagcctcagctgccccagaaatatacagcg

SEQ ID NO: 23 - DNA, 31 bp, Mus musculus
gtggaggaaagtgcagagaaacaggccaaag

SEQ ID NO: 24 - DNA, 30 bp, Mus musculus
ccccaggaaagtctacacctgcagcaagtg

SEQ ID NO: 25 - DNA, 30 bp, Mus musculus
cagcacgtgcgaactcacacaggagaacgc

SEQ ID NO: 26 - DNA, 30 bp, Mus musculus
gtccttactcaagctctcagaagactcatc

SEQ ID NO: 27 - DNA, 28 bp, Mus musculus
atgtggcctctaatcagcatgaagtgac

SEQ ID NO: 28 - DNA, 30 bp, Mus musculus
cacaacgggcctaaacctcttaattgcccg

SEQ ID NO: 29 - DNA, 32 bp, Mus musculus
aacccacggcagttcaactgccccgtgtgtga

SEQ ID NO: 30 - DNA, 25 bp, Mus musculus
ctaagaagtgtaatctacaatacca

SEQ ID NO: 31 - DNA, 27 bp, Mus musculus
caaatctaagcatcccacctgtcccag

SEQ ID NO: 32 - DNA, 25 bp, Mus musculus
aataacgccgtcagcaacgagaaga

SEQ ID NO: 33 - DNA, 30 bp, Mus musculus
cccttaagaaaggcacaaagaagacgccgc

SEQ ID NO: 34 - DNA, 30 bp, Mus musculus
caggcagaggtcaccgggtcaggatcttct

SEQ ID NO: 35 - DNA, 30 bp, Mus musculus
cccagaaggaaccacctcccagtatggagc

SEQ ID NO: 36 - DNA, 30 bp, Mus musculus
ggcttggtgcctgttagagacagcaagctt

SEQ ID NO: 37 - DNA, 29 bp, Mus musculus
gtgacgtggacactgagcagaagacagat

SEQ ID NO: 38 - RNA, 30 nt, Mus musculus
aaccaacgacauguacgaccugcacgagcu

SEQ ID NO: 39 - RNA, 31 nt, Mus musculus
gaagccucagcugccccagaaauauacagcg

SEQ ID NO: 40 - RNA, 31 nt, Mus musculus
guggaggaaagugcagagaaacaggccaaag

SEQ ID NO: 41 - RNA, 30 nt, Mus musculus
ccccaggaaagucuacaccugcagcaagug

SEQ ID NO: 42 - RNA, 30 nt, Mus musculus
cagcacgugcgaacucacacaggagaacgc

SEQ ID NO: 43 - RNA, 30 nt, Mus musculus
guccuuacucaagcucucagaagacucauc

SEQ ID NO: 44 - RNA, 28 nt, Mus musculus
auguggccucuaaucagcaugaagugac

SEQ ID NO: 45 - RNA, 30 nt, Mus musculus
cacaacgggccuaaaccucuuaauugcccg

SEQ ID NO: 46 - RNA, 32 nt, Mus musculus
aacccacggcaguucaacugccccguguguga

SEQ ID NO: 47 - RNA, 25 nt, Mus musculus
cuaagaaguguaaucuacaauacca

SEQ ID NO: 48 - RNA, 27 nt, Mus musculus
caaaucuaagcaucccaccugucccag

SEQ ID NO: 49 - RNA, 25 nt, Mus musculus
aauaacgccgucagcaacgagaaga

SEQ ID NO: 50 - RNA, 30 nt, Mus musculus
cccuuaagaaaggcacaaagaagacgccgc

SEQ ID NO: 51 - RNA, 30 nt, Mus musculus
caggcagaggucaccgggucaggaucuucu

SEQ ID NO: 52 - RNA, 30 nt, Mus musculus
cccagaaggaaccaccucccaguauggagc

SEQ ID NO: 53 - RNA, 30 nt, Mus musculus
ggcuuggugccuguuagagacagcaagcuu

SEQ ID NO: 54 - RNA, 29 nt, Mus musculus
gugacguggacacugagcagaagacagau

SEQ ID NO: 55 - DNA, 20 bp, Artificial Sequence
  gRNA
ggcgcagcagcagaagaccg

SEQ ID NO: 56 - DNA, 20 bp, Artificial Sequence
  gRNA
accgcagcgacggcagaacc

SEQ ID NO: 57 - DNA, 20 bp, Artificial Sequence
  gRNA
ccctggttctgccgtcgctg

SEQ ID NO: 58 - DNA, 20 bp, Artificial Sequence
  gRNA
agcgacggcagaaccagggc

SEQ ID NO: 59 - DNA, 20 bp, Artificial Sequence
  gRNA
cgggatcagaccgccggccc

SEQ ID NO: 60 - DNA, 20 bp, Artificial Sequence
  gRNA
gatcgcaccccgggatctcg

SEQ ID NO: 61 - DNA, 20 bp, Artificial Sequence
  gRNA
gagttggagcggcggcgacg

SEQ ID NO: 62 - DNA, 20 bp, Artificial Sequence
  gRNA
atactgtggctcgggcggcg

SEQ ID NO: 63 - DNA, 20 bp, Mus musculus
ggcgcagcagcagaagaccg

SEQ ID NO: 64 - DNA, 20 bp, Mus musculus
accgcagcgacggcagaacc

SEQ ID NO: 65 - DNA, 20 bp, Mus musculus
ccctggttctgccgtcgctg

SEQ ID NO: 66 - DNA, 20 bp, Mus musculus
agcgacggcagaaccagggc

SEQ ID NO: 67 - DNA, 20 bp, Mus musculus
cgggatcagaccgccggccc

SEQ ID NO: 68 - DNA, 20 bp, Mus musculus
gatcgcaccccgggatctcg

SEQ ID NO: 69 - DNA, 20 bp, Mus musculus
gagttggagcggcggcgacg

SEQ ID NO: 70 - DNA, 20 bp, Mus musculus
atactgtggctcgggcggcg

SEQ ID NO: 71 - DNA, 21 bp, Artificial Sequence
  gRNA
ggcgggcggcgacggcgcggg

SEQ ID NO: 72 - DNA, 21 bp, Artificial Sequence
  gRNA
gcgcggcgcagcgtcctgtgc

SEQ ID NO: 73 - DNA, 21 bp, Artificial Sequence
  gRNA
agcgacggcagaaccagggcc

SEQ ID NO: 74 - DNA, 21 bp, Artificial Sequence
  gRNA
cggccctggttctgccgtcgc

SEQ ID NO: 75 - DNA, 21 bp, Artificial Sequence
  gRNA
ggaccgtgggcgcacagttca

SEQ ID NO: 76 - DNA, 21 bp, Artificial Sequence
  gRNA
cggccgccgcgccgcccgagc

SEQ ID NO: 77 - DNA, 21 bp, Mus musculus
ggcgggcggcgacggcgcggg

SEQ ID NO: 78 - DNA, 21 bp, Mus musculus
gcgcggcgcagcgtcctgtgc

SEQ ID NO: 79 - DNA, 21 bp, Mus musculus
agcgacggcagaaccagggcc

SEQ ID NO: 80 - DNA, 21 bp, Mus musculus
cggccctggttctgccgtcgc

SEQ ID NO: 81 - DNA, 21 bp, Mus musculus
ggaccgtgggcgcacagttca

SEQ ID NO: 82 - DNA, 21 bp, Mus musculus
cggccgccgcgccgcccgagc

SEQ ID NO: 83 - RNA, 30 nt, Artificial Sequence
  gRNA
uaagcugaggugcggccaguucagcuuugg

SEQ ID NO: 84 - RNA, 30 nt, Artificial Sequence
  gRNA
ucacagcagcugccauuuacuuccccaguu

SEQ ID NO: 85 - RNA, 27 nt, Artificial Sequence
  gRNA
ccaucugucuuucuucaccgaccaggu

SEQ ID NO: 86 - RNA, 29 nt, Artificial Sequence
  gRNA
cuguaaauaucuggagcaccugaugccuc

SEQ ID NO: 87 - RNA, 30 nt, Artificial Sequence
  gRNA
uucugcuucauauuggcauggcuuacagcg

SEQ ID NO: 88 - RNA, 30 nt, Artificial Sequence
  gRNA
cuuagcacugugaacucugaugugaugcac

SEQ ID NO: 89 - RNA, 28 nt, Artificial Sequence
  gRNA
uugccugcuucucugcacucucuuccac

SEQ ID NO: 90 - RNA, 30 nt, Artificial Sequence
  gRNA
cagcggucacagcgaauggggcccuuggag

SEQ ID NO: 91 - RNA, 31 nt, Artificial Sequence
  gRNA
cucauuauccccagcucuggugugguguuuc

SEQ ID NO: 92 - RNA, 28 nt, Artificial Sequence
  gRNA
ugauacucgcucacuguuguguaugugc

SEQ ID NO: 93 - RNA, 29 nt, Artificial Sequence
  gRNA
cguucuccuguaugaguucuaacaugcug

SEQ ID NO: 94 - RNA, 29 nt, Artificial Sequence
  gRNA
augagucuucugagaacuugaguaaggac

SEQ ID NO: 95 - RNA, 32 nt, Artificial Sequence
  gRNA
ucuaguuagaugagucuucugagaacuugagu

SEQ ID NO: 96 - RNA, 29 nt, Artificial Sequence
  gRNA
cauaugucuaguuagaugagucuucugag

SEQ ID NO: 97 - RNA, 28 nt, Artificial Sequence
  gRNA
aaugaguacgcauaugucuaguuagaug

SEQ ID NO: 98 - RNA, 30 nt, Artificial Sequence
  gRNA
cuucucaccugaaugaguacgcauaugucu

SEQ ID NO: 99 - RNA, 31 nt, Artificial Sequence
  gRNA
cacugaucacauuuaaauggcuucucaccug

SEQ ID NO: 100 - RNA, 28 nt, Artificial Sequence
  gRNA
aggccacauaacugcacugaucacauuu

SEQ ID NO: 101 - RNA, 28 nt, Artificial Sequence
  gRNA
guuacuucauguugauuagaggccacau

SEQ ID NO: 102 - RNA, 31 nt, Artificial Sequence
  gRNA
cccauugugaaccugucuugcauggcggguu

SEQ ID NO: 103 - RNA, 30 nt, Artificial Sequence
  gRNA
ugggcaauuaagagguuuaggcccauugug

SEQ ID NO: 104 - RNA, 29 nt, Artificial Sequence
  gRNA
uaaucacagugugggcaauuaagagguuu

SEQ ID NO: 105 - RNA, 28 nt, Artificial Sequence
  gRNA
auuaggacaaguaggaugcuuagauuug

SEQ ID NO: 106 - RNA, 29 nt, Artificial Sequence
  gRNA
aagauuccucaucauucacaggaccaugu

SEQ ID NO: 107 - RNA, 30 nt, Artificial Sequence
  gRNA
agcaggccccuccugaaccaccuccaucug

SEQ ID NO: 108 - RNA, 30 nt, Artificial Sequence
  gRNA
cagcagcuccuucugagcaggccccuccug

SEQ ID NO: 109 - RNA, 30 nt, Artificial Sequence
  gRNA
auuugggcaaccuccgucugagcagucucc

SEQ ID NO: 110 - RNA, 28 nt, Artificial Sequence
  gRNA
ccucuaugggaggagacagcucuaucuu

SEQ ID NO: 111 - RNA, 29 nt, Artificial Sequence
  gRNA
cuaucuuuaacaggcacuaagccaacuuc

SEQ ID NO: 112 - RNA, 32 nt, Artificial Sequence
  gRNA
cucucaucugccucuucugcuccuacuuucug

SEQ ID NO: 113 - RNA, 30 nt, Artificial Sequence
  gRNA
ccauuuaaaguuucacccucuggcguauuc

SEQ ID NO: 114 - RNA, 29 nt, Artificial Sequence
  gRNA
cuauacugucagucugauguuuaccauuu

SEQ ID NO: 115 - RNA, 32 nt, Artificial Sequence
  gRNA
uucuucuacugcugaagggggaagcauuggug

SEQ ID NO: 116 - RNA, 29 nt, Artificial Sequence
  gRNA
caggaggugaugccagugcaguuuuggac

SEQ ID NO: 117 - RNA, 29 nt, Artificial Sequence
  gRNA
uggaacuggccgagccccaugcaauccag

SEQ ID NO: 118 - RNA, 29 nt, Artificial Sequence
  gRNA
cugaaagaacgaucacagaagauacaaac

SEQ ID NO: 119 - DNA, 25 bp, Homo sapiens
ccaaagcgaacggccgcacccagca

SEQ ID NO: 120 - DNA, 30 bp, Homo sapiens
aactggggaagtaaatggcagctgctgtga

SEQ ID NO: 121 - DNA, 27 bp, Homo sapiens
acctggtcggtgaagaaagacagatgg

SEQ ID NO: 122 - DNA, 29 bp, Homo sapiens
gaggcatcaggtgctccagatatttacag

SEQ ID NO: 123 - DNA, 30 bp, Homo sapiens
cgctgtaagccatgccaatatgaagcagaa

SEQ ID NO: 124 - DNA, 30 bp, Homo sapiens
gtgcatcacatcagagttcacagtgctaag

SEQ ID NO: 125 - DNA, 28 bp, Homo sapiens
gtggaagagagtgcagagaagcaggcaa

SEQ ID NO: 126 - DNA, 30 bp, Homo sapiens
ctccaagggccccattcgctgtgaccgctg

SEQ ID NO: 127 - DNA, 31 bp, Homo sapiens
gaaacaccacaccagagctggggataatgag

SEQ ID NO: 128 - DNA, 28 bp, Homo sapiens
gcacatacacaacagtgagcgagtatca

SEQ ID NO: 129 - DNA, 29 bp, Homo sapiens
cagcatgttagaactcatacaggagaacg

SEQ ID NO: 130 - DNA, 29 bp, Homo sapiens
gtccttactcaagttctcagaagactcat

SEQ ID NO: 131 - DNA, 32 bp, Homo sapiens
actcaagttctcagaagactcatctaactaga

SEQ ID NO: 132 - DNA, 29 bp, Homo sapiens
ctcagaagactcatctaactagacatatg

SEQ ID NO: 133 - DNA, 28 bp, Homo sapiens
catctaactagacatatgcgtactcatt

SEQ ID NO: 134 - DNA, 30 bp, Homo sapiens
agacatatgcgtactcattcaggtgagaag

SEQ ID NO: 135 - DNA, 31 bp, Homo sapiens
caggtgagaagccatttaaatgtgatcagtg

SEQ ID NO: 136 - DNA, 28 bp, Homo sapiens
aaatgtgatcagtgcagttatgtggcct

SEQ ID NO: 137 - DNA, 28 bp, Homo sapiens
atgtggcctctaatcaacatgaagtaac

SEQ ID NO: 138 - DNA, 31 bp, Homo sapiens
aacccgccatgcaagacaggttcacaatggg

SEQ ID NO: 139 - DNA, 30 bp, Homo sapiens
cacaatgggcctaaacctcttaattgccca

SEQ ID NO: 140 - DNA, 29 bp, Homo sapiens
aaacctcttaattgcccacactgtgatta

SEQ ID NO: 141 - DNA, 28 bp, Homo sapiens
caaatctaagcatcctacttgtcctaat

SEQ ID NO: 142 - DNA, 29 bp, Homo sapiens
acatggtcctgtgaatgatgaggaatctt

SEQ ID NO: 143 - DNA, 30 bp, Homo sapiens
cagatggaggtggttcaggaggggcctgct

SEQ ID NO: 144 - DNA, 30 bp, Homo sapiens
caggaggggcctgctcagaaggagctgctg

SEQ ID NO: 145 - DNA, 30 bp, Homo sapiens
ggagactgctcagacggaggttgcccaaat

SEQ ID NO: 146 - DNA, 28 bp, Homo sapiens
aagatagagctgtctcctcccatagagg

SEQ ID NO: 147 - DNA, 29 bp, Homo sapiens
gaagttggcttagtgcctgttaaagatag

SEQ ID NO: 148 - DNA, 32 bp, Homo sapiens
cagaaagtaggagcagaagaggcagatgagag

SEQ ID NO: 149 - DNA, 30 bp, Homo sapiens
gaatacgccagagggtgaaactttaaatgg

SEQ ID NO: 150 - DNA, 29 bp, Homo sapiens
aaatggtaaacatcagactgacagtatag

SEQ ID NO: 151 - DNA, 32 bp, Homo sapiens
caccaatgcttcccccttcagcagtagaagaa

SEQ ID NO: 152 - DNA, 29 bp, Homo sapiens
gtccaaaactgcactggcatcacctcctg

SEQ ID NO: 153 - DNA, 29 bp, Homo sapiens
ctggattgcatggggctcggccagttcca

SEQ ID NO: 154 - DNA, 29 bp, Homo sapiens
gtttgtatcttctgtgatcgttctttcag

SEQ ID NO: 155 - RNA, 30 nt, Homo sapiens
ccaaagcugaacuggccgcaccucagcuua

SEQ ID NO: 156 - RNA, 30 nt, Homo sapiens
aacuggggaaguaaauggcagcugcuguga

SEQ ID NO: 157 - RNA, 27 nt, Homo sapiens
accuggucggugaagaaagacagaugg

SEQ ID NO: 158 - RNA, 29 nt, Homo sapiens
gaggcaucaggugcuccagauauuuacag

SEQ ID NO: 159 - RNA, 30 nt, Homo sapiens
cgcuguaagccaugccaauaugaagcagaa

SEQ ID NO: 160 - RNA, 30 nt, Homo sapiens
gugcaucacaucagaguucacagugcuaag

SEQ ID NO: 161 - RNA, 28 nt, Homo sapiens
guggaagagagugcagagaagcaggcaa

SEQ ID NO: 162 - RNA, 30 nt, Homo sapiens
cuccaagggccccauucgcugugaccgcug

SEQ ID NO: 163 - RNA, 31 nt, Homo sapiens
gaaacaccacaccagagcuggggauaaugag

SEQ ID NO: 164 - RNA, 28 nt, Homo sapiens
gcacauacacaacagugagcgaguauca

SEQ ID NO: 165 - RNA, 29 nt, Homo sapiens
cagcauguuagaacucauacaggagaacg

SEQ ID NO: 166 - RNA, 29 nt, Homo sapiens
guccuuacucaaguucucagaagacucau

SEQ ID NO: 167 - RNA, 32 nt, Homo sapiens
acucaaguucucagaagacucaucuaacuaga

SEQ ID NO: 168 - RNA, 29 nt, Homo sapiens
cucagaagacucaucuaacuagacauaug

SEQ ID NO: 169 - RNA, 28 nt, Homo sapiens
caucuaacuagacauaugcguacucauu

SEQ ID NO: 170 - RNA, 30 nt, Homo sapiens
agacauaugcguacucauucaggugagaag

SEQ ID NO: 171 - RNA, 31 nt, Homo sapiens
caggugagaagccauuuaaaugugaucagug

SEQ ID NO: 172 - RNA, 28 nt, Homo sapiens
aaaugugaucagugcaguuauguggccu

SEQ ID NO: 173 - RNA, 28 nt, Homo sapiens
auguggccucuaaucaacaugaaguaac

SEQ ID NO: 174 - RNA, 31 nt, Homo sapiens
aacccgccaugcaagacagguucacaauggg

SEQ ID NO: 175 - RNA, 30 nt, Homo sapiens
cacaaugggccuaaaccucuuaauugccca

SEQ ID NO: 176 - RNA, 29 nt, Homo sapiens
aaaccucuuaauugcccacacugugauua

SEQ ID NO: 177 - RNA, 28 nt, Homo sapiens
caaaucuaagcauccuacuuguccuaau

SEQ ID NO: 178 - RNA, 29 nt, Homo sapiens
acaugguccugugaaugaugaggaaucuu

SEQ ID NO: 179 - RNA, 30 nt, Homo sapiens
cagauggaggugguucaggaggggccugcu

SEQ ID NO: 180 - RNA, 30 nt, Homo sapiens
caggaggggccugcucagaaggagcugcug

SEQ ID NO: 181 - RNA, 30 nt, Homo sapiens
ggagacugcucagacggagguugcccaaau

SEQ ID NO: 182 - RNA, 28 nt, Homo sapiens
aagauagagcugucuccucccauagagg

SEQ ID NO: 183 - RNA, 29 nt, Homo sapiens
gaaguuggcuuagugccuguuaaagauag

SEQ ID NO: 184 - RNA, 32 nt, Homo sapiens
cagaaaguaggagcagaagaggcagaugagag

SEQ ID NO: 185 - RNA, 30 nt, Homo sapiens
gaauacgccagagggugaaacuuuaaaugg

SEQ ID NO: 186 - RNA, 29 nt, Homo sapiens
aaaugguaaacaucagacugacaguauag

SEQ ID NO: 187 - RNA, 32 nt, Homo sapiens
caccaaugcuucccccuucagcaguagaagaa

SEQ ID NO: 188 - RNA, 29 nt, Homo sapiens
guccaaaacugcacuggcaucaccuccug

SEQ ID NO: 189 - RNA, 29 nt, Homo sapiens
cuggauugcauggggcucggccaguucca

SEQ ID NO: 190 - RNA, 29 nt, Homo sapiens
guuuguaucuucugugaucguucuuucag

SEQ ID NO: 191 - protein, 1082 aa, Mus musculus
MATQVMGQSSGGGSLFNNSANMGMALTNDMYDLHELSKAELAAPQLIMLANVALTGEASGSCCDYLVGEERQMAELMPVGDNHFSESEGEGLEESADLKGLENMELGSLELSAVEPQPVFEASAAPEIYSANKDPAPETPVAEDKCRSSKAKPFRCKPCQYEAESEEQFVHHIRIHSAKKFFVEESAEKQAKAWESGSSPAEEGEFSKGPIRCDRCGYNTNRYDHYMAHLKHHLRAGENERIYKCIICTYTTVSEYHWRKHLRNHFPRKVYTCSKCNYFSDRKNNYVQHVRTHTGERPYKCELCPYSSSQKTHLTRHMRTHSGEKPFKCDQCNYVASNQHEVTRHARQVHNGPKPLNCPHCDYKTADRSNFKKHVELHVNPRQFNCPVCDYAASKKCNLQYHFKSKHPTCPSKTMDVSKVKLKKTKKREADLLNNAVSNEKMENEQTKTKGDVSGKKNEKPVKAVGKDASKEKKPGSSVSVVQVTTRTRKSAVAAETKAAEVKHTDGQTGNNPEKPCKAKKNKRKKDAEAHPSEEPVNEGPVTKKKKKSECKSKIGTNVPKGGGRAEERPGVKKQSASLKKGTKKTPPKTKTSKKGGKLAPKGMGQTEPSSGALAQVGVSPDPALIQAEVTGSGSSQTELPSPMDIAKSEPAQMEVSLTGPPPVEPAQMEPSPAKPPQVEAPTYPQPPQRGPAPPTGPAPPTGPAPPTEPAPPTGLAEMEPSPTEPSQKEPPPSMEPPCPEELPQAEPPPMEDCQKELPSPVEPAQIEVAQTAPTQVQEEPPPVSEPPRVKPTKRSSLRKDRAEKELSLLSEMARQEQVLMGVGLVPVRDSKLLKGNKSAQDPPAPPSPSPKGNSREETPKDQEMVSDGEGTIVFPLKKGGPEEAGESPAELAALKESARVSSSEQNSAMPEGGASHSKCQTGSSGLCDVDTEQKTDTVPMKDSAAEPVSPPTPTVDRDAGSPAVVASPPITLAENESQEIDEDEGIHSHDGSDLSDNMSEGSDDSGLHGARPTPPEATSKNGKAGLAGKVTEGEFVCIFCDRSFRKEKDYSKHLNRHLVNVYFLEEAAEEQEEQEEREEQE

SEQ ID NO: 192 - DNA, 3249 bp, Mus musculus
atggccacccaggtgatggggcagtcttctggaggaggcagtctcttcaacaacagtgccaacatgggcatggccttaaccaacgacatgtacgacctgcacgagctctcgaaagctgaactggcagcccctcagctcatcatgttagccaacgtggccctgacgggggaggcaagcggcagctgctgcgattacctggtcggtgaagagaggcagatggccgaattgatgcccgtgggagacaaccacttctcagaaagtgaaggagaaggcctggaagagtcggctgacctcaaagggctggaaaacatggaactgggaagtttggagctaagtgctgtagaaccccagcccgtatttgaagcctcagctgccccagaaatatacagcgccaataaagatcccgctccagaaacacccgtggcggaagacaaatgcaggagttctaaggccaagcccttccggtgtaagccttgccagtacgaagccgaatctgaagagcagtttgtgcatcacatccggattcacagcgctaagaagttctttgtggaggaaagtgcagagaaacaggccaaagcctgggagtcggggtcgtctccggccgaagagggcgagttctccaaaggccccatccgctgtgaccgctgtggctacaataccaaccggtatgaccactacatggcacacctgaagcaccacctgcgagctggcgagaacgagcgcatctacaagtgcatcatctgcacgtacacgacggtcagcgagtaccactggaggaaacacctgagaaaccatttccccaggaaagtctacacctgcagcaagtgcaactacttctcagacagaaaaaataactacgttcagcacgtgcgaactcacacaggagaacgcccgtataaatgtgaactttgtccttactcaagctctcagaagactcatctaacgcgacacatgcggactcattcaggtgagaagccatttaaatgtgatcagtgcaattatgtggcctctaatcagcatgaagtgacccgacatgcaagacaggttcacaacgggcctaaacctcttaattgcccgcactgtgactacaaaacagcagatagaagcaacttcaaaaagcacgtggagctgcatgttaacccacggcagttcaactgccccgtgtgtgactacgcggcttctaagaagtgtaatctacaataccatttcaaatctaagcatcccacctgtcccagcaaaacaatggatgtctccaaagtgaagctaaagaaaaccaaaaagagagaggctgacctgcttaataacgccgtcagcaacgagaagatggagaatgagcaaacaaaaacaaagggggatgtgtctgggaagaagaacgagaaacctgtaaaagctgtgggaaaagatgcttcaaaagagaagaagcctggtagcagtgtctcagtggtccaggtaactaccaggactcggaagtcagcggtggcggcggagactaaagcagcagaggtgaaacacacagacggacaaacaggaaacaatccagaaaagccctgtaaagccaagaaaaacaaaagaaagaaggatgctgaggcccatccctccgaagagcctgtgaacgagggaccagtgacaaaaaagaaaaagaagtctgagtgcaaatcaaaaatcggtaccaacgtgccaaagggcggcggccgagcggaggagaggccgggggtcaagaagcaaagcgcttcccttaagaaaggcacaaagaagacgccgcccaagacaaagacaagtaaaaaaggtggcaaacttgctccaaaggggatggggcagacagaaccttcttctggggcattggctcaagtgggggtgtctccagaccctgccctcattcaggcagaggtcaccgggtcaggatcttctcagacagagcttccttcacccatggatattgctaagtcagagcccgcccagatggaggtttccctaacagggccacctccggtggagcctgctcaaatggagccatcgcctgcgaaacctccccaggtagaagcacccacttacccccagcctccccaaagggggcctgcccctcccacggggcctgcccctcccacggggcctgcccctcccacggagcctgcccctcccacggggcttgccgagatggaaccttctcccacggagccttcccagaaggaaccacctcccagtatggagcctccctgccccgaggagctgcctcaggccgagccacctcctatggaggattgtcagaaggagctgccttctcccgtggagcccgctcagattgaggttgctcagacggcccctacgcaggttcaggaggagccccctcctgtctcggagccacctcgggtgaagccaaccaaaagatcatctctccggaaagacagagcagagaaggagctgagcctgctgagtgagatggcgcggcaggagcaggtcctcatgggggttggcttggtgcctgttagagacagcaagcttctgaagggaaacaagagcgcccaggaccccccagccccaccgtcaccatcgccaaagggaaactcgagggaagagacacccaaggaccaagaaatggtctctgatggggaaggaactatagtattccctctcaagaaaggaggaccagaggaagctggagagagtccagctgagttggctgctctcaaggagtctgcccgtgtttcatcctctgaacaaaactcagccatgccagagggtggagcatcacacagcaagtgtcagactggctcctctgggctttgtgacgtggacactgagcagaagacagatactgtccccatgaaagactccgcagcagagccagtgtcccctcctaccccaacagtggaccgtgacgcagggtcaccagctgtagtggcctcccctcctatcacgttggctgaaaacgagtctcaggaaattgatgaagatgaaggcatccatagccatgatggaagtgacctgagtgacaacatgtctgaggggagtgacgactcaggactgcacggggctcggccgacaccaccagaagctacgtcaaaaaatgggaaggcagggttggctggtaaagtgactgagggagagtttgtgtgtattttctgtgatcgttcttttagaaaggaaaaagattatagcaaacacctcaatcgccacttggtgaatgtgtacttcctagaagaagcagctgaggagcaggaggagcaggaggagcgggaggagcaggagtag

SEQ ID NO: 193 - DNA, 20 bp, Artificial Sequence
  Primer
ctggctcttccactgcagaa

SEQ ID NO: 194 - DNA, 20 bp, Artificial Sequence
  Primer
tggtgtttcaggtgtgctgt

SEQ ID NO: 195 - DNA, 20 bp, Artificial Sequence
  Primer
ctacccccaatgtgtccgtc

SEQ ID NO: 196 - DNA, 20 bp, Artificial Sequence
  Primer
aagtcgcaggagacaacctg

SEQ ID NO: 197 - DNA, 20 bp, Artificial Sequence
  Primer
gttagaactcatacaggaga

SEQ ID NO: 198 - DNA, 20 bp, Artificial Sequence
  Primer
gaggtttaggcccattgtga

SEQ ID NO: 199 - DNA, 22 bp, Artificial Sequence
  Primer
gtctcctctgacttcaacagcg

SEQ ID NO: 200 - DNA, 22 bp, Artificial Sequence
  Primer
accaccctgttgctgtagccaa

SEQ ID NO: 201 - DNA, 20 bp, Artificial Sequence
  Primer
ggtcaccagggctgctttta

SEQ ID NO: 202 - DNA, 20 bp, Artificial Sequence
  Primer
ttcccgttctcagccttcac